SEQUENCE LISTING (127 sequences)

SEQ ID NO: 1 - protein, 12 aa, Homo sapiens
CQQQGLPRAAGG

SEQ ID NO: 2 - protein, 12 aa, Homo sapiens
CQQLGLPRAAGG

SEQ ID NO: 3 - protein, 12 aa, Homo sapiens
CQQQGLPRAAEG

SEQ ID NO: 4 - protein, 12 aa, Homo sapiens
CQQLGLPRAAEG

SEQ ID NO: 5 - protein, 11 aa, Homo sapiens
CQQQGLPRAAG

SEQ ID NO: 6 - protein, 11 aa, Homo sapiens
CQQLGLPRAAG

SEQ ID NO: 7 - protein, 11 aa, Homo sapiens
CQQQGLPRAAE

SEQ ID NO: 8 - protein, 11 aa, Homo sapiens
CQQLGLPRAAE

SEQ ID NO: 9 - protein, 15 aa, Homo sapiens
HSGQQQGLPRAAGGC

SEQ ID NO: 10 - protein, 15 aa, Homo sapiens
CHSGQQLGLPRAAGG

SEQ ID NO: 11 - protein, 15 aa, Homo sapiens
CHSGQQQGLPRAAEG

SEQ ID NO: 12 - protein, 15 aa, Homo sapiens
CHSGQQLGLPRAAEG

SEQ ID NO: 13 - protein, 14 aa, Homo sapiens
QSQRAPDRVLCHSG

SEQ ID NO: 14 - protein, 14 aa, Homo sapiens
GSAQSQRAPDRVLC

SEQ ID NO: 15 - protein, 13 aa, Homo sapiens
CGAGRADWPGPPE

SEQ ID NO: 16 - protein, 15 aa, Homo sapiens
CAGRADWPGPPELDV

SEQ ID NO: 17 - protein, 12 aa, Homo sapiens
CGGWPGPPELDV

SEQ ID NO: 18 - protein, 21 aa, Homo sapiens
CHSGQQQGLPRAAGGSVPHPR

SEQ ID NO: 19 - protein, 21 aa, Homo sapiens
HSGQQQGLPRAAGGSVPHPRC

SEQ ID NO: 20 - protein, 38 aa, Homo sapiens
GLAGGSAQSQRAPDRVLCHSGQQQGLPRAAGGSVPHPR

SEQ ID NO: 21 - protein, 26 aa, Homo sapiens
GLAGGSAQSQRAPDRVLCHSGQQQGL

SEQ ID NO: 22 - protein, 17 aa, Homo sapiens
PELDVCVEEAEGEAPWT

SEQ ID NO: 23 - protein, 15 aa, Homo sapiens
ELDVCVEEAEGEAPW

SEQ ID NO: 24 - protein, 15 aa, Homo sapiens
TQLLCVEAFEGEEPW

SEQ ID NO: 25 - protein, 16 aa, Homo sapiens
RADWPGPPELDVCVEE

SEQ ID NO: 26 - protein, 8 aa, Homo sapiens
RADWPGPP

SEQ ID NO: 27 - protein, 21 aa, Homo sapiens
SVNPGLAGGSAQSQRAPDRVL

SEQ ID NO: 28 - protein, 22 aa, Homo sapiens
SVNPGLAGGSAQSQRAPDRVLC

SEQ ID NO: 29 - protein, 20 aa, Homo sapiens
HSGQQQGLPRAAGGSVPHPR

SEQ ID NO: 30 - protein, 12 aa, Homo sapiens
CGAGRADWPGPP

SEQ ID NO: 31 - protein, 11 aa, Homo sapiens
GAGRADWPGPP

SEQ ID NO: 32 - protein, 17 aa, Homo sapiens
GLAGGSAQSQRAPDRVL

SEQ ID NO: 33 - protein, 17 aa, Homo sapiens
GPPELDVCVEEAEGEAP

SEQ ID NO: 34 - protein, 10 aa, Homo sapiens
GLPRAAGGSV

SEQ ID NO: 35 - protein, 10 aa, Homo sapiens
HSGQQQGLPR

SEQ ID NO: 36 - protein, 10 aa, Homo sapiens
PRAAGGSVPH

SEQ ID NO: 37 - protein, 9 aa, Homo sapiens
LPRAAGGSV

SEQ ID NO: 38 - protein, 9 aa, Homo sapiens
RAAGGSVPH

SEQ ID NO: 39 - protein, 10 aa, Homo sapiens
RVLCHSGQQQ

SEQ ID NO: 40 - protein, 9 aa, Homo sapiens
GLAGGSAQS

SEQ ID NO: 41 - protein, 10 aa, Homo sapiens
QRAPDRVLCH

SEQ ID NO: 42 - protein, 9 aa, Homo sapiens
SQRAPDRVL

SEQ ID NO: 43 - protein, 9 aa, Homo sapiens
RAPDRVLCH

SEQ ID NO: 44 - protein, 9 aa, Homo sapiens
QRAPDRVLC

SEQ ID NO: 45 - protein, 9 aa, Homo sapiens
WPGPPELDV

SEQ ID NO: 46 - protein, 9 aa, Homo sapiens
GPPELDVCV

SEQ ID NO: 47 - protein, 9 aa, Homo sapiens
WPGPPELDV

SEQ ID NO: 48 - protein, 9 aa, Homo sapiens
QQQGLPRAA

SEQ ID NO: 49 - protein, 9 aa, Homo sapiens
QQGLPRAAG

SEQ ID NO: 50 - protein, 9 aa, Homo sapiens
QGLPRAAGG

SEQ ID NO: 51 - protein, 9 aa, Homo sapiens
GPPELDVCV

SEQ ID NO: 52 - protein, 9 aa, Homo sapiens
QQLGLPRAA

SEQ ID NO: 53 - protein, 9 aa, Homo sapiens
QLGLPRAAG

SEQ ID NO: 54 - protein, 9 aa, Homo sapiens
LGLPRAAGG

SEQ ID NO: 55 - protein, 9 aa, Homo sapiens
QQGLPRAAE

SEQ ID NO: 56 - protein, 9 aa, Homo sapiens
QGLPRAAEG

SEQ ID NO: 57 - protein, 10 aa, Homo sapiens
HSGQQQGLPR

SEQ ID NO: 58 - protein, 9 aa, Homo sapiens
GLPRAAGGC

SEQ ID NO: 59 - protein, 9 aa, Homo sapiens
SGQQQGLPR

SEQ ID NO: 60 - protein, 9 aa, Homo sapiens
SQRAPDRVL

SEQ ID NO: 61 - protein, 8 aa, Homo sapiens
QRAPDRVL

SEQ ID NO: 62 - protein, 10 aa, Homo sapiens
QRAPDRVLCH

SEQ ID NO: 63 - protein, 8 aa, Homo sapiens
QRAPDRVL

SEQ ID NO: 64 - protein, 9 aa, Homo sapiens
SQRAPDRVL

SEQ ID NO: 65 - protein, 9 aa, Homo sapiens
QRAPDRVLC

SEQ ID NO: 66 - protein, 13 aa, Homo sapiens
RAVSVNPGLAGGC

SEQ ID NO: 67 - protein, 13 aa, Homo sapiens
AVSVNPGLAGGSC

SEQ ID NO: 68 - protein, 13 aa, Homo sapiens
VSVNPGLAGGSAC

SEQ ID NO: 69 - protein, 13 aa, Homo sapiens
SVNPGLAGGSAQC

SEQ ID NO: 70 - protein, 13 aa, Homo sapiens
VNPGLAGGSAQSC

SEQ ID NO: 71 - protein, 13 aa, Homo sapiens
NPGLAGGSAQSQC

SEQ ID NO: 72 - protein, 12 aa, Homo sapiens
GLAGGSAQSQRC

SEQ ID NO: 73 - protein, 15 aa, Homo sapiens
CGLAGGSAQSQRAPD

SEQ ID NO: 74 - protein, 12 aa, Homo sapiens
CGGAQSQRAPDR

SEQ ID NO: 75 - protein, 12 aa, Homo sapiens
AQSQRAPDRGGC

SEQ ID NO: 76 - protein, 13 aa, Homo sapiens
CSAQSQRAPDRVL

SEQ ID NO: 77 - protein, 13 aa, Homo sapiens
SAQSQRAPDRVLC

SEQ ID NO: 78 - protein, 12 aa, Homo sapiens
CGGSQRAPDRVL

SEQ ID NO: 79 - protein, 15 aa, Homo sapiens
APDRVLCHSGQQQGC

SEQ ID NO: 80 - protein, 14 aa, Homo sapiens
RVLCHSGQQQGLPR

SEQ ID NO: 81 - protein, 15 aa, Homo sapiens
CQQQGLPRAAGGSVP

SEQ ID NO: 82 - protein, 14 aa, Homo sapiens
LPRAAGGSVPHPRC

SEQ ID NO: 83 - protein, 14 aa, Homo sapiens
AAGGSVPHPRCHAG

SEQ ID NO: 84 - protein, 14 aa, Homo sapiens
CVPHPRAHAGAGRA

SEQ ID NO: 85 - protein, 14 aa, Homo sapiens
HPRAHCGAGRADWP

SEQ ID NO: 86 - protein, 12 aa, Homo sapiens
WPGPPELDVGGC

SEQ ID NO: 87 - protein, 14 aa, Homo sapiens
DWPGPPELDVCVEE

SEQ ID NO: 88 - protein, 13 aa, Homo sapiens
PPELDVCVEEAEG

SEQ ID NO: 89 - protein, 13 aa, Homo sapiens
CGGLDVAVEEAEG

SEQ ID NO: 90 - protein, 14 aa, Homo sapiens
DVAVEEAEGEAGGC

SEQ ID NO: 91 - protein, 14 aa, Homo sapiens
LDVCVEEAEGEAPW

SEQ ID NO: 92 - protein, 11 aa, Homo sapiens
CVEEAEGEAPW

SEQ ID NO: 93 - protein, 14 aa, Homo sapiens
HSGQQLGLPRAAGC

SEQ ID NO: 94 - protein, 12 aa, Homo sapiens
CQQQGLPRAAGG

SEQ ID NO: 95 - protein, 16 aa, Homo sapiens
HSGQQQGLPRAAGGCK

SEQ ID NO: 96 - protein, 67 aa, Homo sapiens
AVSVNPGLAGGSAQSQRAPDRVLCHSGQQQGLPRAAGGSVPHPRCHCGAGRADWPGPPELDVCVEEK

SEQ ID NO: 97 - protein, 24 aa, Homo sapiens
CHSGQQQGLPRAAGGSVPHPRCHK

SEQ ID NO: 98 - protein, 24 aa, Homo sapiens
CHSGQQQGLPRAAGGSVPHPRCHK

SEQ ID NO: 99 - protein, 12 aa, Homo sapiens
CQQIGLPRAAGG

SEQ ID NO: 100 - protein, 12 aa, Homo sapiens
CQQVGLPRAAGG

SEQ ID NO: 101 - protein, 12 aa, Homo sapiens
CQQFGLPRAAGG

SEQ ID NO: 102 - protein, 12 aa, Homo sapiens
CQQMGLPRAAGG

SEQ ID NO: 103 - protein, 12 aa, Homo sapiens
CQQNGLPRAAGG

SEQ ID NO: 104 - protein, 12 aa, Homo sapiens
CQQAGLPRAAGG

SEQ ID NO: 105 - protein, 12 aa, Homo sapiens
CQQGGLPRAAGG

SEQ ID NO: 106 - protein, 12 aa, Homo sapiens
CQQSGLPRAAGG

SEQ ID NO: 107 - protein, 12 aa, Homo sapiens
CQQTGLPRAAGG

SEQ ID NO: 108 - protein, 12 aa, Homo sapiens
CQQPGLPRAAGG

SEQ ID NO: 109 - protein, 11 aa, Homo sapiens
QQQGLPRAAGG

SEQ ID NO: 110 - protein, 11 aa, Homo sapiens
QQLGLPRAAGG

SEQ ID NO: 111 - protein, 11 aa, Homo sapiens
QQQGLPRAAEG

SEQ ID NO: 112 - protein, 11 aa, Homo sapiens
QQLGLPRAAEG

SEQ ID NO: 113 - protein, 10 aa, Homo sapiens
QQQGLPRAAG

SEQ ID NO: 114 - protein, 10 aa, Homo sapiens
QQLGLPRAAG

SEQ ID NO: 115 - protein, 10 aa, Homo sapiens
QQQGLPRAAE

SEQ ID NO: 116 - protein, 10 aa, Homo sapiens
QQLGLPRAAE

SEQ ID NO: 117 - protein, 14 aa, Homo sapiens
HSGQQQGLPRAAGG

SEQ ID NO: 118 - protein, 14 aa, Homo sapiens
HSGQQLGLPRAAGG

SEQ ID NO: 119 - protein, 14 aa, Homo sapiens
HSGQQQGLPRAAEG

SEQ ID NO: 120 - protein, 14 aa, Homo sapiens
HSGQQLGLPRAAEG

SEQ ID NO: 121 - protein, 14 aa, Homo sapiens
QSQRAPDRVLCHSG

SEQ ID NO: 122 - protein, 13 aa, Homo sapiens
GSAQSQRAPDRVL

SEQ ID NO: 123 - protein, 12 aa, Homo sapiens
GAGRADWPGPPE

SEQ ID NO: 124 - protein, 14 aa, Homo sapiens
AGRADWPGPPELDV

SEQ ID NO: 125 - protein, 9 aa, Homo sapiens
WPGPPELDV

SEQ ID NO: 126 - protein, 65 aa, Homo sapiens
GLAGGSAQSQRAPDRVLCHSGQQQGLPRAAGGSVPHPRCHCGAGRADWPGPPELDVCVEEAEGEA

SEQ ID NO: 127 - protein, 12 aa, homo sapiens
CLAGQGRQPQGA

(19) United States
(12) Patent Application Publication
(10) Pub. No.: US 2018/0186896 A1
(43) Pub. Date: Jul. 5, 2018

(54) VACCINES FOR THE TREATMENT AND PREVENTION OF IGE MEDIATED DISEASES

(71) Applicant: AFFIRIS AG, Vienna (AT)

(72) Inventors: Oskar SMRZKA, Vienna (AT); Benjamin VIGL, Vienna (AT)

(73) Assignee: AFFIRIS AG, Vienna (AT)

(21) Appl. No.: 15/738,759

(22) PCT Filed: Jul. 7, 2016
(86) PCT No.: PCT/EP2016/066111

§ 371 Date: Dec. 21, 2017

(30) Foreign Application Priority Data
Jul. 7, 2015 (EP) 15175562.6

(51) Int. Cl. C07K 16/42; A61K 39/00

(52) U.S. Cl. CPC C07K 16/4291; A61K 39/0005; A61K 2039/505; A61K 2039/627; A61K 2039/70; C07K 2317/21; C07K 2317/34; A61K 2039/6081

(57) ABSTRACT

Disclosed is a vaccine for use in the prevention or treatment of an Immunoglobulin E (IgE-) related disease, comprising a peptide bound to a pharmaceutically acceptable carrier, wherein said peptide is selected from the group of QQQGLPRAAGG (SEQ ID No. 109; p9347), QQLGLPRAAGG (SEQ ID No. 110; p8599), QQQGLPRAAEG (SEQ ID No. I11; p8600), QQLGLPRAAEG (SEQ ID No. 112; p8601), QQQGLPRAAG (SEQ ID No. 113; p9338), QQLGLPRAAG (SEQ ID No. 114; p9041), QQQGLPRAAE (SEQ ID No. 115; p9042), QQLGLPRAAE (SEQ ID No. 116; p9043), HSGQQQGLPRAAGG (SEQ ID No. 117; p7575), HSGQQLGLPRAAGG (SEQ ID No. 118; p8596), HSGQQQGLPRAAEG (SEQ ID No. 119; p8597), HSGQQLGLPRAAEG (SEQ ID No. 120; p8598), QSQRAPDRVLCHSG (SEQ ID No. 121; p7580), GSAQSQRAPDRVL (SEQ ID No. 122; p7577), and WPGPPELDV (SEQ ID No. 125; p7585).

The present invention relates to active vaccination for the treatment and prevention of IgE related diseases as product patent.

IgE mediates immediate hypersensitivity reactions to minute amounts of allergen in sensitized individuals. The efficacy of allergic reactions is based on the local presence of IgE, on the upregulation of high affinity IgE receptor on mast cells in the mucosa and on the exceptionally slow dissociation of IgE from its receptor. However the rarest immunoglobulin isotype constitutes not only the “allergen-receptor” but it also plays a role in parasite infections, tumor immunity and autoimmune diseases. With the advent of clinical anti-IgE trials in a variety of allergic diseases and comorbidities, a whole range of IgE-dependent and IgE-related diseases are being identified [Holgate 2014]. In industrialized societies, the prevalence of allergies is currently reaching 10-30%. As a consequence, extensive effort has been devoted to developing new drugs that target the IgE pathway and in particular the IgE molecule per se. More recently, evidence has turned up that IgE might also play a role in extended areas of inflammation- and allergy-related diseases including chronic urticaria, atopic dermatitis, allergic gastroenteropathy and various (auto)immune-mediated conditions [Holgate 2014]. Thus, therapeutic and preventive IgE targeting has been recognized as a major challenge for a growing number of diseases. In consequence, there is an increasing demand for affordable and broadly applicable anti-IgE therapeutics.

IgE exists predominantly as soluble plasma protein or as receptor bound protein captured by its high affinity IgE-receptor on e.g. mast cells or basophils or low affinity receptors. Alternatively, the molecule is found as B cell receptor (i.e. the IgE-BCR) on rare, IgE-switched cells such as membrane IgE positive B cells that will eventually differentiate to IgE-producing plasma cells upon antigen or allergen stimulus. Correspondingly, receptor-bound IgE mediates the allergic response on effector cells such as e.g. mast cells, whereas the IgE-BCR is a membrane-integrated receptor required for either B cell stimulation or suppression, depending on the presence or absence of co-stimulatory signals, respectively.

In allergy, soluble plasma IgE recognizes multivalent allergens through its variable region and binds to the IgE receptor through its constant chain. As a consequence, IgE-receptor signalling mediates organ-specific and systemic allergic reactions via cells carrying the IgE receptor. Blocking of the IgE/IgE-receptor interaction by the prototypic anti-IgE antibody Omalizumab® thus efficiently reduces plasma IgE levels and thereby alleviates clinical symptoms in allergy patients [Milgrom 1999]. There is a requirement for very high affinity when targeting IgE/IgE-receptor competition. On the other hand high specificity is required in order to restrict IgE binding to the soluble but not to the receptor-bound form of IgE present e.g. on basophils and mast cells which might trigger undesired anaphylaxia. With the avenue of Omalizumab®, this targeting principle has grown to a well validated, therapeutically and commercially successful therapeutic approach for the treatment of severe, therapy resistant asthma. At the same time, the IgE targeting field is expanding with a growing number of off-label exploratory trials with Omalizumab® [Incorvaia 2014]. It is expected that second generation therapeutic anti-IgE antibodies featuring improved efficacy and pharmaceutical characteristics will rapidly progress to new IgE-related, clinical indications [Holgate 2014].

Despite its success, several limitations have prevented Omalizumab® from being applied for a broader range of IgE-related indications. This includes application in paediatric conditions, food allergy, milder manifestations of allergy such as allergic rhinoconjunctivitis and mild forms of allergic asthma or at the other extreme, applications in very high IgE-diseases. Cost of goods for therapeutic antibodies are generally high and require e.g. for Omalizumab® a biweekly 375 mg s.c. injection for a 70-80 kg patient with 400-500 IU/ml IgE plasma levels. Because of such doses, the drug is not approved for very high IgE patients or heavy and overweight patients and not affordable for a broad disease such as allergic rhinoconjunctivits. Other reasons for restricted use include an unfavourable risk to benefit ratio in certain conditions such as food allergy, lack of efficacy or patient compliance or simply the lack of efficacy in a subgroup of asthma patients. Per definition, passively administered anti-soluble IgE antibodies such as Omalizumab® require intrinsically high dosing in order to fulfil pharmacodynamic requirements.

It is not expected that modifications of Omalizumab® dosing schemes will significantly alleviate dosing restrictions for current anti-IgE therapy or lower the financial burden [Lowe et al 2015]. Because of these limitations, an alternative IgE targeting mechanisms addressing IgE supply rather than receptor/ligand interaction has been developed and validated: In contrast to soluble IgE, the membrane form of IgE represents the IgE-BCR. This form is generated by an alternatively spliced extension at the 3′ end of the IgE heavy chain transcript expressed in differentiating, IgE-switched cells [reviewed by Achatz 2008]. Alternative splicing encodes an extended variant of the protein containing three additional domains located C-terminally of the fourth immunoglobulin domain encompassing the so called Extracellular Membrane Proximal Domain (EMPD) followed by the transmembrane and the intracellular domain of the receptor molecule. The IgE-EMPD is unique to the IgE-BCR and therefore present only on IgE switched B cells. Signalling via the IgE-BCR will eventually lead to differentiation of B cells into IgE-producing plasma cells which in turn will fuel IgE-mediated allergic reactions in a positive feedback loop.

It has previously been shown that crosslinking of BCR induces apoptosis [Benhamou 1990] and that a similar concept might be exploited for therapeutic purpose in e.g. allergy when applying antibodies that crosslink the IgE-BCR in order to suppress IgE production [Chang 1990; Haba 1990]. Based on this proposal, it should be feasible to target antibodies by passive or active immunization against components of membrane IgE that will not react with soluble IgE or IgE immobilized on e.g. mast cells or basophils which would provide a risk for mast cell release reactions and anaphylaxis. In vitro and in vivo proofs of this concept [Inführ et al. 2005] have previously been provided using monoclonal or polyclonal antibodies against the EMPD region of the IgE-BCR in various models [WO 1998/053843 A1; Chen 2002; Feichtner 2008; Brightbill 2010]. Alternatively, it was shown that immune sera from mice that were immunized against membrane IgE-EMPD are able to promote in vitro apoptosis and ADCC in membrane IgE-EMPD expressing cells thereby suggesting that this approach might also be accomplished by active instead of passive immunization (such as previously proposed by Lin et al. 2012; WO 2004/000217 A2; EP 1 972 640 A1; US 2014/0220042 A1).

The concept of addressing the IgE-BCR by active vaccination against the IgE EMPD region was further proposed in early days e.g. in U.S. Pat. No. 5,274,075 A, WO 1996/012740 A1 and WO 1998/053843 A1. The initial idea was that in absence of co-stimulatory signals, crosslinking of the IgE-BCR ultimately leads to inhibition of IgE production by various cellular mechanisms [Wu 2014]. Additional cellular mechanisms might contribute to the in vivo mode of action of the IgE-BCR targeting strategy. These mechanisms include anergy [Batista 1996], apoptosis [Poggianella 2006], complement-dependent cytolysis [Chen 2002] or Antibody Dependent Cellular Cytotoxicity (ADCC) [Chen 2010]. In conclusion, IgE EMPD targeting efficiently reduces plasma IgE as demonstrated in allergic conditions [Gauvreau 2014]. In contrast to soluble IgE targeting (e.g. with Omalizumab®), membrane IgE targeting addresses IgE supply rather than the effector function via its receptor or clearance of free plasma IgE.

WO 2010/097012 A1 discloses anti-CεmX antibodies binding to human m/gE on β lymphocytes. WO 2008/116149 A2 refers to apoptotic anti-IgE antibodies. WO 69/12740 A1 discloses synthetic IgE membrane anchor peptide immunogens for the treatment of allergy.

Despite the success of antibody therapeutics, a general concern of passive immunization remains the induction of anti-drug antibodies (ADA's) when using recombinant large therapeutic molecules such as antibodies or related scaffolds. Per definition, anti-IgE therapies require long term treatment with repeated dosing. At the same time, the risk of ADA induction becomes particularly relevant when a large amount of recombinant protein must be repeatedly administered over a longer treatment period. To date, the risk of ADA induction against large protein therapeutics cannot reliably be predicted in particular when recombinant biopharmaceuticals tend to aggregate when mixed with human plasma. As a consequence, extensive clinical trials would be required and at the same time, an open discussion about the problems caused by anti-drug antibodies (ADAs) and the causes and consequences of immunogenicity of modern biologics is restricted by commercial and strategic interests from industry [Deehan 2015]. T cell immunogenicity, on the other hand, requires stringent preclinical assessment [Jawa 2013]. In addition, the cost of goods for large biologicals continues to pose a challenge for public health systems especially if a biological drug such as e.g. a monoclonal antibody should be applied for “milder” indications such as allergic rhinitis and conjunctivitis or non-allergic conditions such as e.g. chronic urticaria where the IgE pathway plays a contributing role in pathogenesis.

It is an object of the present invention to provide an efficient, cost-effective, safe and long lasting prevention or treatment regime for all types of IgE-mediated diseases, especially also for those diseases that are currently not treated with passive immunization due to cost reasons, patient compliance or adverse effects due to injection of a recombinant biological drug such as a humanized monoclonal antibody. On the other hand, if active immunization is chosen as such regime, there is also the desire that cytotoxic and helper T cell reactions against the target per se are avoided in order to eliminate the risk of autoimmune-like adverse effects. The regime must be specific on the disease whereas normal immunological performance of the patient's immune system should not be hampered by the administration of the drug.

Therefore, the present invention provides a vaccine for use in the prevention or treatment of an Immunoglobulin E (IgE-) related disease, comprising at least one peptide bound to a pharmaceutically acceptable carrier, wherein said peptide is selected from the group of QQQGLPRAAGG (SEQ ID No. 109; p9347), QQLGLPRAAGG (SEQ ID No. 110; p8599), QQQGLPRAAEG (SEQ ID No. 111; p8600), QQLGLPRAAEG (SEQ ID No. 112; p8601), QQQGLPRAAG (SEQ ID No. 113; p9338), QQLGLPRAAG (SEQ ID No. 114; p9041), QQQGLPRAAE (SEQ ID No. 115; p9042), QQLGLPRAAE (SEQ ID No. 116; p9043), HSGQQQGLPRAAGG (SEQ ID No. 117; p7575), HSGQQLGLPRAAGG (SEQ ID No. 118; p8596), HSGQQQGLPRAAEG (SEQ ID No. 119; p8597), HSGQQLGLPRAAEG (SEQ ID No. 120; p8598), QSQRAPDRVLCHSG (SEQ ID No. 121; p7580), GSAQSQRAPDRVL (SEQ ID No. 122; p7577), and WPGPPELDV (SEQ ID No. 125; p7585) (hereinafter referred to as the “peptides of the present invention” or the “present peptides”).

The peptides according to the present invention are used for active anti-EMPD vaccination for the treatment and prevention of IgE related diseases. IgE-related disease include allergic diseases such as seasonal, food, pollen, mold spores, poison plants, medication/drug, insect-, scorpion- or spider-venom, latex or dust allergies, pet allergies, allergic asthma bronchiale, non-allergic asthma, Churg-Strauss Syndrome, allergic rhinitis and -conjunctivitis, atopic dermatitis, nasal polyposis, Kimura's disease, contact dermatitis to adhesives, antimicrobials, fragrances, hair dye, metals, rubber components, topical medicaments, rosins, waxes, polishes, cement and leather, chronic rhinosinusitis, atopic eczema, autoimmune diseases where IgE plays a role (“autoallergies”), chronic (idiopathic) and autoimmune urticaria, cholinergic urticaria, mastocytosis, especially cutaneous mastocytosis, allergic bronchopulmonary aspergillosis, chronic or recurrent idiopathic angioedema, interstitial cystitis, anaphylaxis, especially idiopathic and exercise-induced anaphylaxis, immunotherapy, eosinophil-associated diseases such as eosinophilic asthma, eosinophilic gastroenteritis, eosinophilic otitis media and eosinophilic oesophagitis (see e.g. Holgate 2014, U.S. Pat. No. 8,741,294 B2, Usatine 2010). Furthermore the peptides according to the present invention are used for the treatment of lymphomas or the prevention of sensibilisation side effects of an anti-acidic treatment, especially for gastric or duodenal ulcer or reflux. For the present invention, the term “IgE-related disease” includes or is used synonymously to the terms “IgE-dependent disease” or “IgE-mediated disease”.

In response to the limitations of passively administered biologicals, the present invention therefore provides a safe, active vaccination approach. According to the present invention an anti-IgE EMPD response is induced in a patient that provides long lasting IgE suppression. In contrast to close-meshed passive immunization protocols, active immunization requires fewer injections at lower costs. The advantage of a “therapeutic” or “preventive” active vaccination approach is to exploit the body's own humoral immune response in order to avoid administration of large amounts of “foreign”, recombinant protein or biopharmaceuticals that might induce undesired anti-drug antibodies (ADAs) because of their molecular size and antigenicity. Furthermore safety preconditions require a vaccine formulation that strictly limits anti-IgE EMPD immunity to the humoral system—i.e. vaccine induced antibodies—while avoiding cytotoxic or helper T cell reactions against IgE EMPD. In this context, it was previously proposed to use a hepatitis B core antigen-conjugated peptide vaccine for actively inducing an anti-membrane IgE-EMPD targeted immune response [Lin 2012]. This proposal of an active anti-IgE-EMPD vaccine did not take into account safety concerns for autoreactive T cells when addressing IgE-EMPD by active vaccination as a therapeutic modality in IgE-related diseases. Autoreactive T cell induction can e.g. be observed when using peptide vaccination in order to intentionally induce experimental encephalitis in the EAE animal models for multiple sclerosis [Petermann 2011]. Another example for undesired T cell reactions induced by vaccine peptides was e.g. the aborted clinical vaccine trial using T cell epitope containing Abeta peptide [Pride 2008]. To date, the high risk of a possible autoreactive T cell response against IgE EMPD (as a self-antigen) cannot be excluded. Therefore, a vaccine that avoids any type of helper-, cytotoxic- or inhibitory T cell response as the vaccines according to the present invention are clearly favourable compared to prior art proposals: The idea of therapeutic peptide vaccines is to strictly bypass any “natural”, “self” T cell epitopes in order to avoid uncontrollable, autoreactive T cells possibly causing an undesired, autoimmune-like condition. Instead there should be an efficient induction of the humoral immune response producing antibodies that efficiently cross react with the desired target such as IgE EMPD.

In contrast to previously proposed anti-IgE-EMPD active vaccine peptides and proteins, vaccines of the present invention contain shorter peptides that are devoid of any undesired T cell epitopes. Especially in combination with a carrier such as e.g. KLH or CRM or a virosome, a VLP or a polymer based carrier that exposes the B cell epitope in high density in combination with a defined T cell epitope for T cell stimulation. Alternatively particles can be used that include a carrier moiety comprising a liposome, a micelle, or a polymeric nanoparticle (such as proposed in patent WO 2007127221). Essentially they are capable of inducing an anti-EMPD-specific B cell response due to dense exposure of antigenic peptides while T cell help is contributed only by T cell epitopes present on or within the carrier but not on the B cell epitope of the vaccine formulation i.e. the peptide itself of the present invention. If, in such a preferred embodiment (and in contrast to the Virus Like Particles (VLPs) proposed by Lin 2012), peptides are linked via an inert linker to the surface of the carrier instead of being an integrated part of a recombinant VLP protein, no specific and unintended T cell response against IgE is obtained. Furthermore, based on their short size, vaccine peptides of the present invention were developed not to induce undesired off-target responses as observed in the present examples or with prior art antibodies targeting different epitopes of membrane IgE EMPD [Chowdhury 2012].

In conclusion, the present invention proposes specific anti-IgE EMPD vaccine peptides that specifically induce antibody-mediated effector functions such as IgE-BCR crosslinking, ADCC and apoptosis on target cells carrying the IgE-BCR. In contrast to previously proposed vaccines, the present invention provides vaccine peptides that are (1) devoid of T cell epitopes and (2) that lack the increased risk for inducing off-target antibodies while maintaining comparable biologic/cellular activity.

Accordingly (and as extensively shown in the example section below), the peptides according to the present invention are superior as active B cell vaccine than peptides or other EMPD derived protein or peptide sequences incorporated or combined with a carrier protein as previously proposed in the prior art. These superior properties are evident from the example section wherein the superiority of the peptides according to the present invention are compared to prior art vaccine candidates (e.g. Lin et al. 2012; WO 2004/000217 A2; EP 1 972 640 A1; US 2014/0220042 A1). These results show that those prior art proposal are less suited for active B cell vaccination than the peptides according to the present invention.

For example, the peptides according to the present invention are not binding to HLA class I and therefore cannot induce a HLA Class I-restricted cytotoxic T cell response.

Specifically the 11- and 12-mers of the peptides according to the present invention do—per definition—not efficiently bind to HLA class II, because they are too short and therefore will not normally induce a HLA Class II-restricted T helper response.

The peptides according to the present invention are immunogenic and induced antibodies bind better to the membrane IgE-BCR membrane IgE-EMPD than other peptides. The present peptides are safe with respect to inducing off-target effects and antibodies that unspecifically bind to unknown cell surface proteins e.g. from PBMCs in contrast to previously proposed peptides (Lobert, 2013; McIntush, 2013; Ahmed, 2015). The peptides according to the present invention are able to induce an antibody response that mediates functional membrane IgE-BCR crosslinking which induces signalling via the BCR in order to drive cells to apoptosis. Compared to other short peptides derived from the IgE EMPD region, the present peptides are more effective in membrane IgE-BCR crosslinking than and at least as effective as long prior art-derived peptides. Their crosslinking effectivity can be enhanced by combination of two or more short peptides.

The peptides according to the present invention have the potential to induce ADCC/CDC which both contributes to their functional activity (as previously demonstrated for other anti-EMPD antibodies).

The peptides according to the present invention are able to induce antibodies that show affinity to EMPD peptides. This correlates with membrane IgE crosslinking/signal induction in a similar range than antibodies generated by long peptides.

The peptides according to the present invention are able to inhibit IgE secretion from mouse splenocytes derived from transgenic mice carrying a replacement of the endogenous EMPD sequence by human EMPD.

Moreover, the present peptides are able to inhibit IgE secretion from human PBMCs.

The present peptides also comprise peptide variants of the native sequence (“VARIOTOPE®s”) that contain certain amino acid substitutions that provide similar or improved immunogenicity, safety, specificity and functional activity compared to the native sequences. For example, even particular double amino acid substitutions, such as exemplified by p9347 (SEQ ID No. 109), show significantly improved properties compared to the native sequence.

The antibodies elicited by the peptides (and VARIOTOPE®s) according to the present invention are specifically directed against human IgE-EMPD. The main advantage of an active immunization over passive vaccination with monoclonal antibodies lies in the lower cost for the individual and/or the health care system, the presumably longer duration of the immune response after completion of the regimen and the lower probability for the elicitation of anti-drug-antibodies due to the polyclonal nature of the response.

The vaccine according to the present invention is composed of a membrane IgE-specific peptide bound to a pharmaceutically acceptable carrier. This carrier can be directly coupled to the peptides according to the present invention. It is also possible to provide certain linker molecules between the peptide and the carrier. Provision of such linkers may result in beneficial properties of the vaccine, e.g. improved immunogenicity, improved specificity or improved handling (e.g. due to improved solubility or formulation capacities). According to a preferred embodiment, the peptides according to the present invention contain at least one cysteine residue bound as a linker to the N- or C-terminus of the peptide. Although both orientations of the peptide (i.e. N- or C-terminally linked variants) are acceptable for performing the present invention, it may be preferred for some of the peptides to use either the N- or the C-terminal variant because one of these variants may provide advantageous effects (e.g. with respect to HLA binding properties) compared to the other. Specifically preferred examples are the peptides according to SEQ ID Nos. 1 to 14 and 17. This cysteine residue can then be used to covalently couple (“link”) the peptide to the carrier.

Accordingly, in a preferred vaccine according to the present invention the peptide is bound to the carrier by a linker. The linker may be any covalently or non-covalently bound chemical linking moiety that is pharmaceutically suitable and acceptable. According to a preferred embodiment, the linker is a peptide linker, especially a peptide linker having from 1 to 5 amino acid residues. Preferred peptide linkers are those that have been applied and/or approved in vaccine technology; peptide linkers comprising or consisting of Cysteine residues, such as Gly-Gly-Cys, Gly-Gly, Gly-Cys, Cys-Gly and Cys-Gly-Gly, are specifically preferred. Alternatively these peptide linker amino acids can be replaced or combined with charged amino acids in order to guarantee solubility or physically spacing of the peptide epitope from the carrier.

Other preferred linker moieties are chemical coupling molecules that have already been used (and are known to be safe) in pharmaceutical preparations and safeguard an effective linking between the peptide according to the present invention and the pharmaceutically acceptable carrier. Such linkers have also been foreseen in conjugates proposed or used for pharmaceutical preparations as “spacers” to provide spatial distance between two chemical moieties (here: between the peptide and the carrier). For example, bispecific low molecular weight (e.g. MW 500 Da or below, preferably 300 Da or below, especially 100 Da or below) molecules with two different chemically reactive groups (the first being specific for the carrier; the second for the peptide) may be used as linkers. Coupling of the peptide to the carrier by hydrophobic interactions or e.g. with biotin/(strept)avidin systems is also possible.

The present invention also comprises peptide combinations, comprising (a) one or more peptides of the present invention combined with one or more peptide candidates according to the prior art (e.g. IgE peptides (or mIgE-EMPD peptides) that have been suggested in the prior art for the prevention or treatment of IgE-related diseases) or comprising (b) two or more peptides according to the present invention. Preferably, the peptide combination includes two peptides from different regions of IgE (e.g. native amino acid residues 8-21 and/or 22-32, especially a peptide selected from the group QQQGLPRAAGG (SEQ ID No. 109; p9347), QQLGLPRAAGG (SEQ ID No. 110; p8599), QQQGLPRAAEG (SEQ ID No. 111; p8600), and QQLGLPRAAEG (SEQ ID No. 112; p8601), and a peptide from another region of the IgE molecule, especially a peptide selected from the group QSQRAPDRVLCHSG (SEQ ID No. 121; p7580), GSAQSQRAPDRVL (SEQ ID No. 122; p7577), HSGQQQGLPRAAGG (SEQ ID No. 117; p7575), and WPGPPELDV (SEQ ID No. 125; p7585). Specifically preferred are therefore combinations comprising at least one of SEQ ID No. 109, 110, 111, 112, 113, 114, 115, or 116 and SEQ ID No. 117, 121, 122 or 125 (or fragments with a length of 13, 12, 11, 10, 9, 8, 7 or 6 amino acid residues of SEQ ID Nos. 117, 121, 122 or 125), especially a combination comprising SEQ ID Nos. 109 and 121. The present invention also refers to fragments of p7580 (QSQRAPDRVLCHSG; SEQ ID No. 121) with a length of 13, 12, 11, 10, 9, or 8, 7 or 6 amino acid residues of SEQ ID Nos. 121, alone or in a combination with other peptides according to the present invention, especially with suitable linker amino acids or linker peptides, carriers and in the formulations as disclosed herein.

Accordingly, the present peptides have significant distinguishing features in comparison to prior art proposals for IgE vaccines making them superior as active B cell vaccine than previously proposed peptides or other EMPD derived protein or peptide sequence incorporated or combined with a carrier in a vaccine formulation.

The present vaccines contain the peptide(s) according to the present invention in a form wherein the peptide(s) is (are) bound to a pharmaceutically acceptable carrier. According to the present invention, any suitable carrier molecule for carrying the present peptides may be used for the vaccines according to the present invention, as long as this carrier is pharmaceutically acceptable, i.e. as long as it is possible to provide such carrier in a pharmaceutical preparation to be administered to human recipients of such vaccines. Preferred carriers according to the present invention are protein carriers, especially keyhole limpet haemocyanin (KLH), tetanus toxoid (TT), Haemophilus influenzae protein D (protein D), or diphtheria toxin (DT). Preferred carriers are also non-toxic diphtheria toxin mutant, especially CRM 197, CRM 176, CRM 228, CRM 45, CRM 9, CRM 102, CRM 103 and CRM 107 (see e.g. Uchida, 1973), whereby CRM 197 is particularly preferred.

Carrier proteins have a specific advantage compared to other carriers, such as VLP-carriers, because the linked peptides strictly induce B cell responses whereas T cell response is solely contributed by the carrier protein. Moreover the density of carrier coupled peptides provides effective BCR activation for B cell activation and differentiation. This contrasts with the VLP-based vaccine proposed by Lin et al, where the peptide epitope is integrated into a recombinant protein and not necessarily designed to induce solely a B cell response. Integrating of a peptide epitope into a recombinant protein structure implies that the peptide will be structurally constrained which can possibly change its antigenic properties and epitope exposure. Therefore it is preferred to link the peptides of the present invention at only one terminus in order to guarantee structural flexibility of the vaccine peptide.

In addition to conventional carrier proteins such as KLH or CRM etc., it is also possible to use modern scaffolds or cell targeting entities that act via bringing together two or more targets e.g. cells or receptors on these cells, such as antigen presenting cells, T cells and B cells. As pharmaceutically active carriers such entities are able to target and/or stimulate receptors and/or cells involved in e.g. antigen processing, antigen processing, B cell or T cell stimulation. Such (multi-)functional carriers can be provided as fusion proteins or poly-specific entities such as exemplified in Kreutz, 2013 using DC targeting via different targeting moieties such as e.g. AB, scFv, alternative scaffolds such as bi- and multispecific proteins or fusion proteins based on antibodies (Weidle 2014) or natural or alternative scaffolds (Weidle 2013) or blood group antigens, sugars, viruses and parts thereof or receptor ligands such as CD40L that are capable of joining distinct functionalities such as two or even more different types of domains, ligands or receptors in order to trigger immunological events. Liu et al, 2014 for example have used lipophilic albumin-binding entities for the purpose of lymph node targeting. Alternatively Silva et al. 2013 showed the use of nanoparticles for addressing DCs.

The vaccine according to the present invention is a vaccine preparation or composition suitable to be applied to human individuals (in this connection, the terms “vaccine”, “vaccine composition” and “vaccine preparation” are used interchangeably herein and identify a pharmaceutical preparation comprising a peptide according to the present invention bound to a pharmaceutically accepted carrier in combination with an adjuvant).

According to a preferred embodiment, the vaccine according to the present invention is formulated with an adjuvant, preferably wherein the peptide bound to the carrier is adsorbed to alum.

The vaccine according to the present invention is preferably formulated for intravenous, subcutaneous, intradermal or intramuscular administration, especially for subcutaneous or intradermal administration.

The vaccine composition according to the present invention preferably contains the peptide according to the present invention in an amount from 0.1 ng to 10 mg, preferably 10 ng to 1 mg, in particular 100 ng to 100 μg. The vaccines of the present invention may be administered by any suitable mode of application, e.g. i.d., i.v., i.p., i.m., intranasally, orally, subcutaneously, transdermally, intradermally etc. and in any suitable delivery device (O'Hagan et al., Nature Reviews, Drug Discovery 2 (9), (2003), 727-735). Therefore, the vaccine of the present invention is preferably formulated for intravenous, subcutaneous, intradermal or intramuscular administration (see e.g. “Handbook of Pharmaceutical Manufacturing Formulations”, Sarfaraz Niazi, CRC Press Inc, 2004).

The vaccine according to the present invention comprises in a pharmaceutical composition the peptides according to the invention in an amount of from 0.1 ng to 10 mg, preferably 10 ng to 1 mg, in particular 100 ng to 100 μg, or, alternatively, e.g. 100 fmol to 10 μmol, preferably 10 pmol to 1 μmol, in particular 100 pmol to 100 nmol. Typically, the vaccine may also contain auxiliary substances, e.g. buffers, stabilizers etc.

Typically, the vaccine composition of the present invention may also comprise auxiliary substances, e.g. buffers, stabilizers etc. Preferably, such auxiliary substances, e.g. a pharmaceutically acceptable excipient, such as water, buffer and/or stabilizers, are contained in an amount of 0.1 to 99% (weight), more preferred 5 to 80% (weight), especially 10 to 70% (weight). Possible administration regimes include a weekly, biweekly, four-weekly (monthly) or bimonthly treatment for about 1 to 12 months; however, also 2 to 5, especially 3 to 4, initial vaccine administrations (in one or two months), followed by boaster vaccinations 6 to 12 months thereafter or even years thereafter are preferred—besides other regimes already suggested for other vaccines.

According to a preferred embodiment of the present invention the peptide in the vaccine is administered to an individual in an amount of 0.1 ng to 10 mg, preferably of 0.5 to 500 μg, more preferably 1 to 100 μg, per immunization. In a preferred embodiment these amounts refer to all peptides present in the vaccine composition of the present invention. In another preferred embodiment these amounts refer to each single peptides present in the composition. It is of course possible to provide a vaccine in which the various different peptides are present in different or equal amounts. However, the peptides of the present invention may alternatively be administered to an individual in an amount of 0.1 ng to 10 mg, preferably 10 ng to 1 mg, in particular 100 ng to 300 μg/kg body weight (as a single dosage).

The amount of peptides that may be combined with the carrier materials to produce a single dosage form will vary depending upon the host treated and the particular mode of administration. The dose of the composition may vary according to factors such as the disease state, age, sex and weight of the individual, and the ability of antibody to elicit a desired response in the individual. Dosage regime may be adjusted to provide the optimum therapeutic response. For example, several divided doses may be administered daily or the dose may be proportionally reduced as indicated by the exigencies of the therapeutic situation. The dose of the vaccine may also be varied to provide optimum preventative dose response depending upon the circumstances. For instance, the vaccines of the present invention may be administered to an individual at intervals of several days, one or two weeks or even months or years depending always on the level of antibodies induced by the administration of the composition of the present invention.

In a preferred embodiment of the present invention the vaccine composition is applied between 2 and 10, preferably between 2 and 7, even more preferably up to 5 and most preferably up to 4 times. This number of immunizations may lead to a basic immunization. In a particularly preferred embodiment the time interval between the subsequent vaccinations is chosen to be between 2 weeks and 5 years, preferably between 1 month and up to 3 years, more preferably between 2 months and 1.5 years. An exemplified vaccination schedule may comprise 3 to 4 initial vaccinations over a period of 6 to 8 weeks and up to 6 months. Thereafter the vaccination may be repeated every two to ten years. The repeated administration of the vaccines of the present invention may maximize the final effect of a therapeutic vaccination.

According to a preferred embodiment of the present invention the vaccine is formulated with at least one adjuvant.

“Adjuvants” are compounds or a mixture that enhance the immune response to an antigen (i.e. the AFFITOPE®s according to the present invention). Adjuvants may act primarily as a delivery system, primarily as an immune modulator or have strong features of both. Suitable adjuvants include those suitable for use in mammals, including humans.

According to a particular preferred embodiment of the present invention the at least one adjuvant used in the vaccine composition as defined herein is capable to stimulate the innate immune system.

Innate immune responses are mediated by toll-like receptors (TLR's) at cell surfaces and by Nod-LRR proteins (NLR) intracellularly and are mediated by D1 and D0 regions respectively. The innate immune response includes cytokine production in response to TLR activation and activation of Caspase-1 and IL-1β secretion in response to certain NLRs (including Ipaf). This response is independent of specific antigens, but can act as an adjuvant to an adaptive immune response that is antigen specific.

A number of different TLRs have been characterized. These TLRs bind and become activated by different ligands, which in turn are located on different organisms or structures. The development of immunopotentiator compounds that are capable of eliciting responses in specific TLRs is of interest in the art. For example, U.S. Pat. No. 4,666,886 describes certain lipopeptide molecules that are TLR2 agonists. WO 2009/118296, WO 2008/005555, WO 2009/111337 and WO 2009/067081 each describe classes of small molecule agonists of TLR7. WO 2007/040840 and WO 2010/014913 describe TLR7 and TLR8 agonists for treatment of diseases. These various compounds include small molecule immunopotentiators (SMIPs).

The at least one adjuvant capable to stimulate the innate immune system preferably comprises or consists of a Toll-like receptor (TLR) agonist, preferably a TLR1, TLR2, TLR3, TLR4, TLR5, TLR7, TLR8 or TLR9 agonist, particularly preferred a TLR4 agonist.

Agonists of Toll-like receptors are well known in the art. For instance a TLR 2 agonist is Pam3CysSerLys4, peptidoglycan (Ppg), PamCys, a TLR3 agonist is IPH 31XX, a TLR4 agonist is an Aminoalkyl glucosaminide phosphate, E6020, CRX-527, CRX-601, CRX-675, 5D24.D4, RC-527, a TLR7 agonist is Imiquimod, 3M-003, Aldara, 852A, R850, R848, CL097, a TLR8 agonist is 3M-002, a TLR9 agonist is Flagellin, Vaxlmmune, CpG ODN (AVE0675, HYB2093), CYT005-15 AllQbG10, dSLIM.

According to a preferred embodiment of the present invention the TLR agonist is selected from the group consisting of monophosphoryl lipid A (MPL), 3-de-O-acylated monophosphoryl lipid A (3D-MPL), poly I:C, GLA, flagellin, R848, imiquimod and CpG.

The composition of the present invention may comprise MPL. MPL may be synthetically produced MPL or MPL obtainable from natural sources. Of course it is also possible to add to the composition of the present invention chemically modified MPL. Examples of such MPL's are known in the art.

According to a further preferred embodiment of the present invention the at least one adjuvant comprises or consists of a saponin, preferably QS21, a water in oil emulsion and a liposome.

The at least one adjuvant is preferably selected from the group consisting of MF59, AS01, AS02, AS03, AS04, aluminium hydroxide and aluminium phosphate.

Examples of known suitable delivery-system type adjuvants that can be used in humans include, but are not limited to, alum (e.g., aluminium phosphate, aluminium sulfate or aluminium hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

Examples of known suitable immune modulatory type adjuvants that can be used in humans include, but are not limited to saponins extracts from the bark of the Aquilla tree (QS21, Quil A), TLR4 agonists such as MPL (Monophosphoryl Lipid A), 3DMPL (3-O-deacylated MPL) or GLA-AQ, LT/CT mutants, cytokines such as the various interleukins (e.g., IL-2, IL-12) or GM-CSF, and the like.

Examples of known suitable immune modulatory type adjuvants with both delivery and immune modulatory features that can be used in humans include, but are not limited to ISCOMS (see, e.g., Sjölander et al. (1998) J. Leukocyte Biol. 64:713; WO90/03184, WO96/11711, WO 00/48630, WO98/36772, WO00/41720, WO06/134423 and WO07/026,190) or GLA-EM which is a combination of a Toll-like receptor agonists such as a TLR4 agonist and an oil-in-water emulsion.

Further exemplary adjuvants to enhance effectiveness of the vaccine compositions of the present invention include, but are not limited to: (1) oil-in-water emulsion formulations (with or without other specific immunostimulating agents such as muramyl peptides (see below) or bacterial cell wall components), such as for example (a) SAF, containing 10% Squalane, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, Mass.), Abisco® (Isconova, Sweden), or Iscomatrix® (Commonwealth Serum Laboratories, Australia), may be used or particles generated therefrom such as ISCOMs (immunostimulating complexes), which ISCOMS may be devoid of additional detergent e.g. WO00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) (see e.g., GB-2220221, EP-A-0689454), optionally in the substantial absence of alum when used with pneumococcal saccharides (see e.g. WO00/56358); (6) combinations of 3dMPL with, for example, QS21 and/or oil-in-water emulsions (see e.g. EP-A-0835318, EP-A-0735898, EP-A-0761231); (7) a polyoxyethylene ether or a polyoxyethylene ester (see e.g. WO99/52549); (8) a polyoxyethylene sorbitan ester surfactant in combination with an octoxynol (WO01/21207) or a polyoxyethylene alkyl ether or ester surfactant in combination with at least one additional non-ionic surfactant such as an octoxynol (WO01/21152); (9) a saponin and an immunostimulatory oligonucleotide (e.g. a CpG oligonucleotide) (WO 00/62800); (10) an immunostimulant and a particle of metal salt (see e.g. WO00/23105); (11) a saponin and an oil-in-water emulsion e.g. WO99/11241; (12) a saponin (e.g. QS21)+3dMPL+IM2 (optionally+a sterol) e.g. WO98/57659; (13) other substances that act as immunostimulating agents to enhance the efficacy of the composition. Muramyl peptides include N-acetyl-muramyl-L-threonyl-D-isoglutamine (thr-MDP), N-25 acetyl-normnuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine MTP-PE), etc.

Particularly preferred compositions of the present invention comprise as adjuvant an oil-in-water emulsion with or without Toll-like receptor agonists, as well as liposomes and/or saponin-containing adjuvants, with or without Toll-like receptor agonists. The composition of the present invention may also comprise aluminium hydroxide with or without Toll-like receptor agonists as adjuvant.

The present invention is further described by the following examples and the figures, yet without being limited thereto.

The figures show:

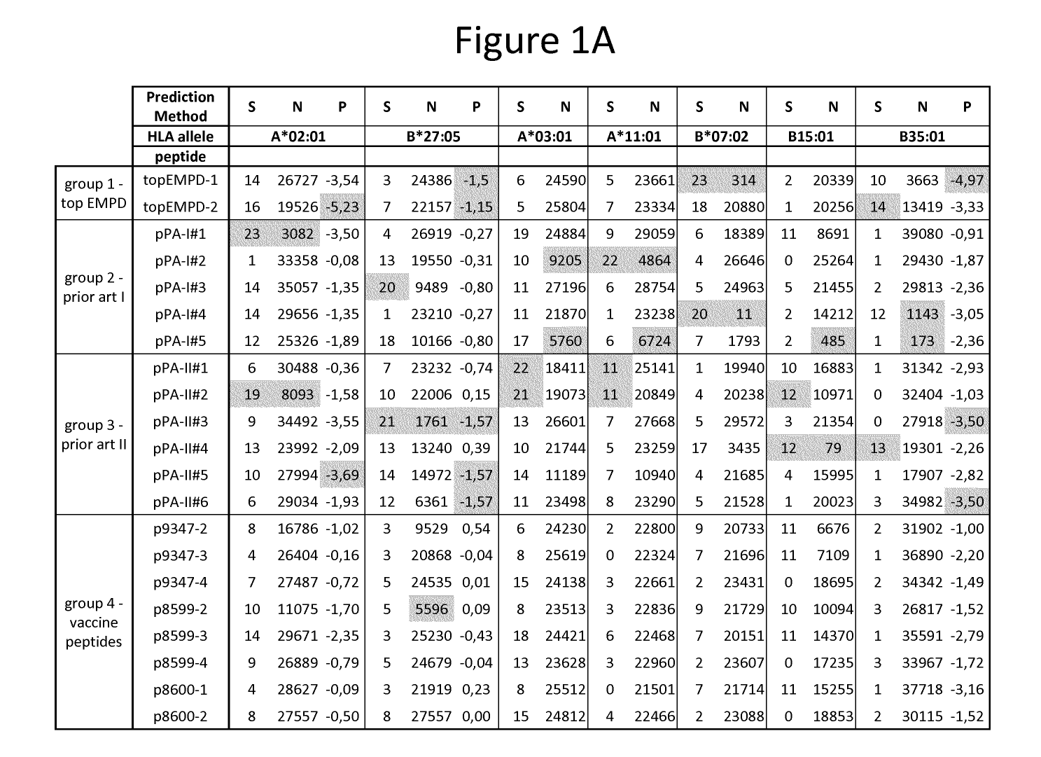

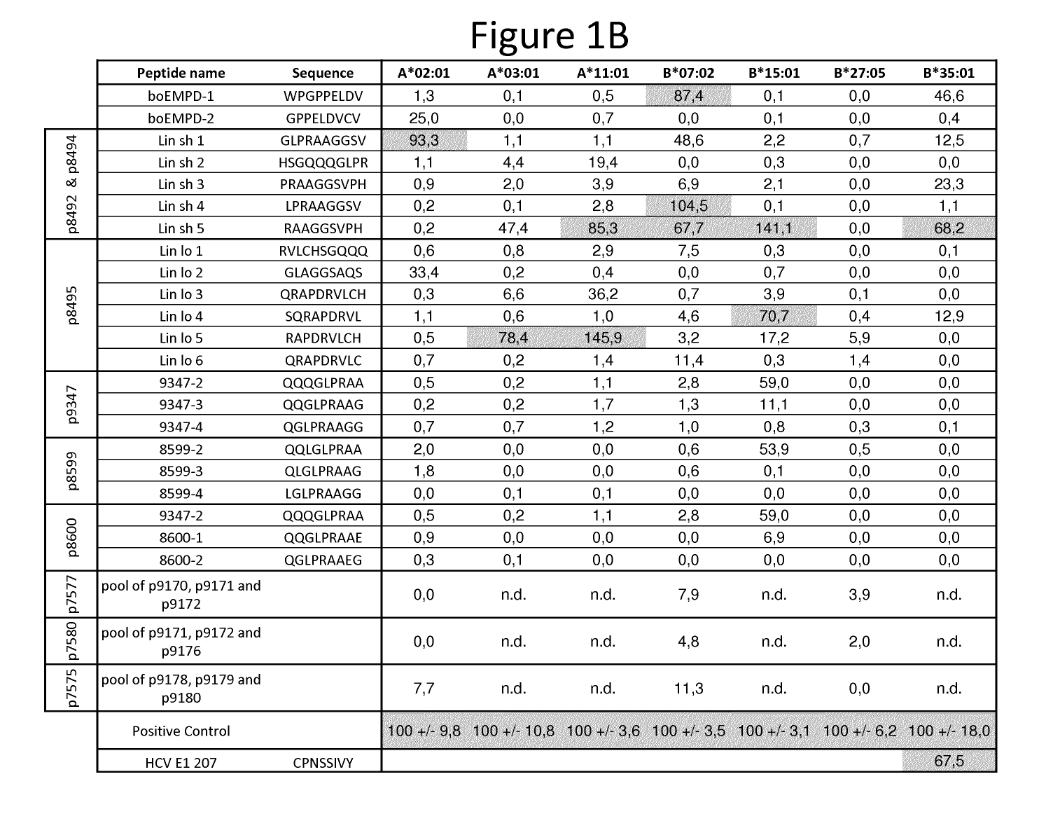

FIG. 1A: Vaccine peptides with a length of 12 or fewer amino acids, starting at position 22 of the human IgE-BCR EMPD region, show lower HLA class I binding prediction scores than e.g. neighboring EMPD derived sequences from previously proposed, active anti membrane IgE EMPD vaccines.

FIG. 1B: Candidate peptides from predictions in FIG. 1A were assembled and analyzed using the REVEAL® HLA class I-peptide binding assay to determine their level of incorporation into HLA molecules.

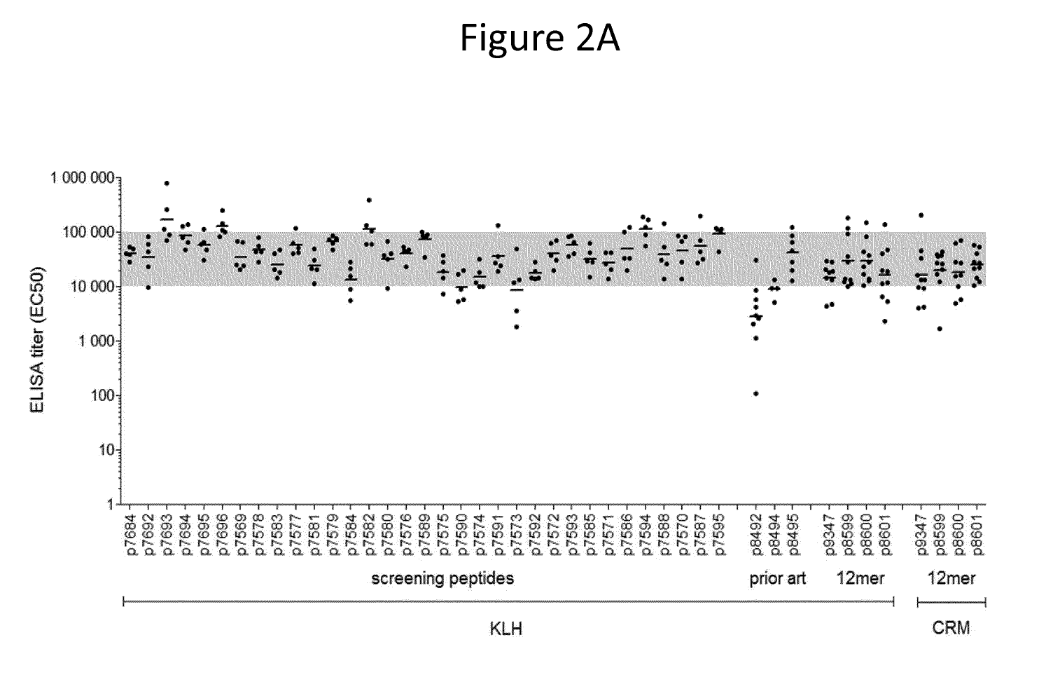

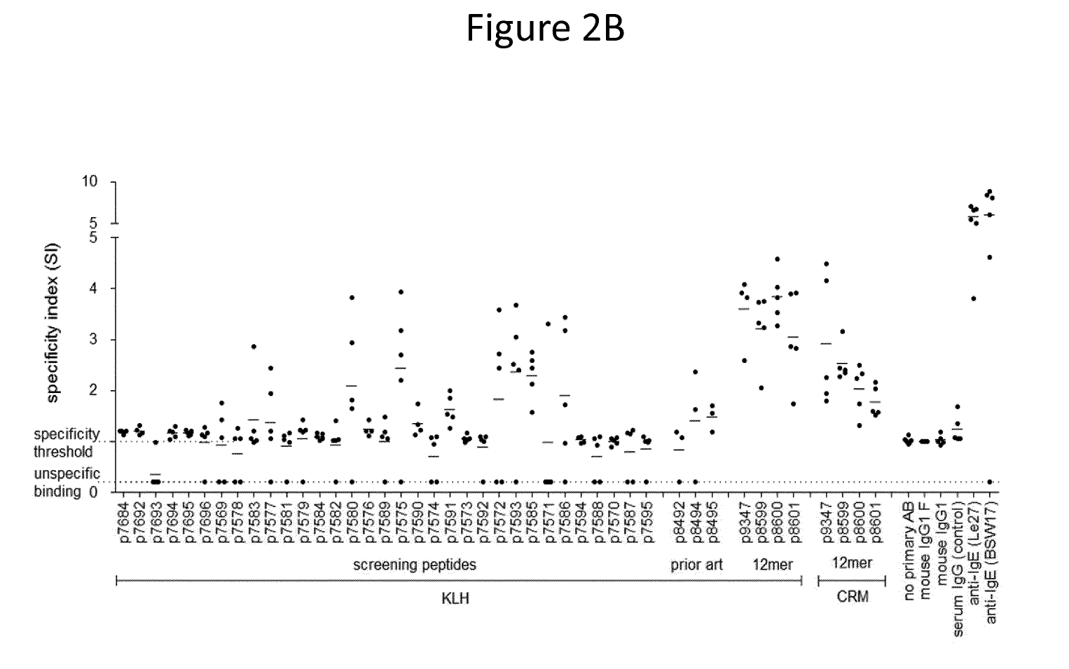

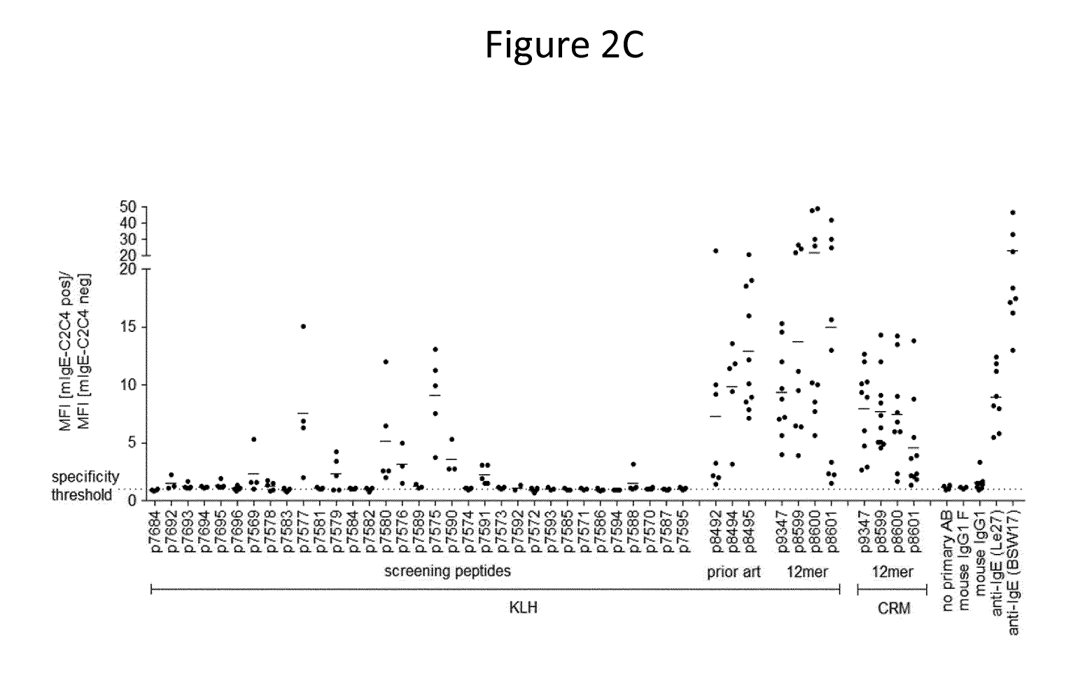

FIG. 2A: All injected peptides are immunogenic.

FIG. 2B: In contrast to their immunogenicity, not all immune sera recognize membrane IgE-EMPD expressed on HEK cells.

FIG. 2C: Membrane IgE-BCR recognition on the cell surface by vaccine-induced antibodies is restricted to few peptide vaccines.

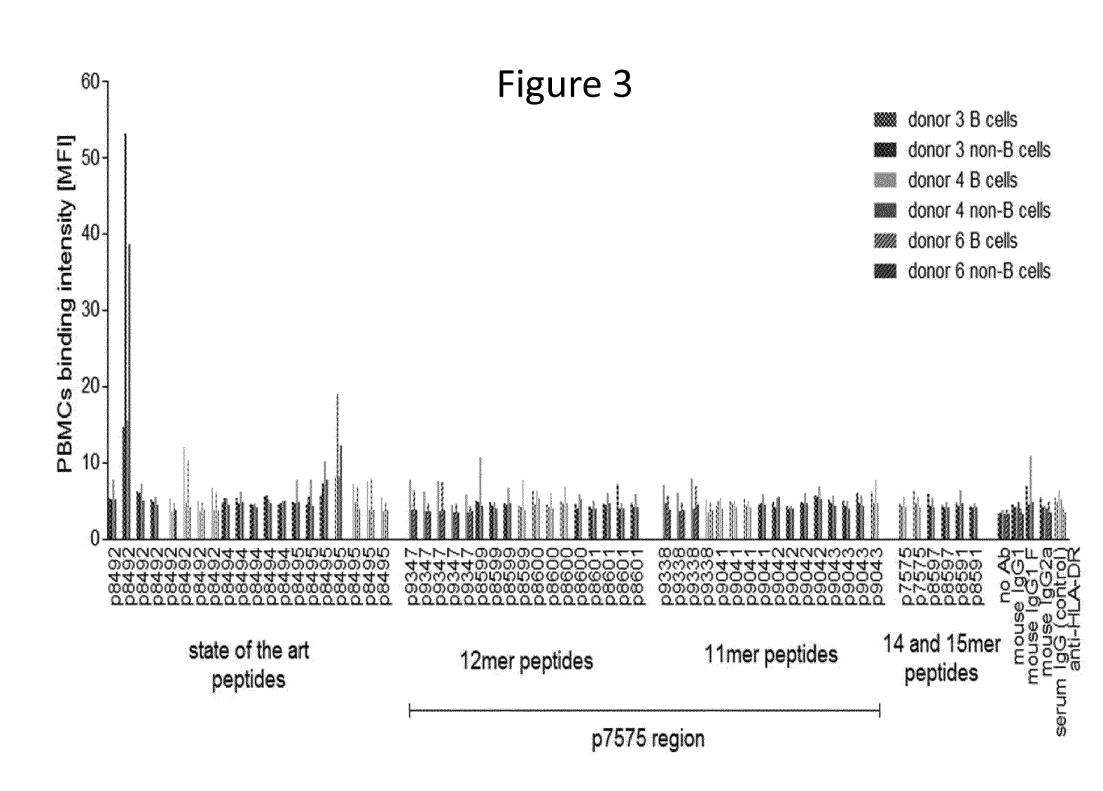

FIG. 3: Peptides of the present invention induce IgE EMPD-specific antibodies that, in contrast to previously proposed active vaccines, do not show unspecific off-target binding to human PBMCs.

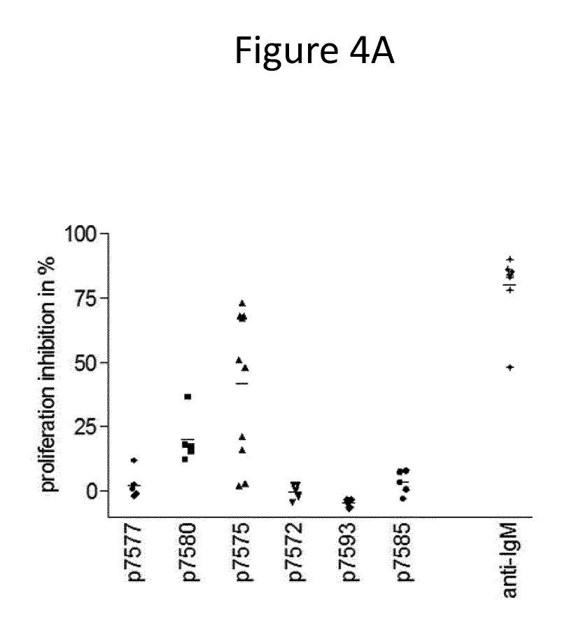

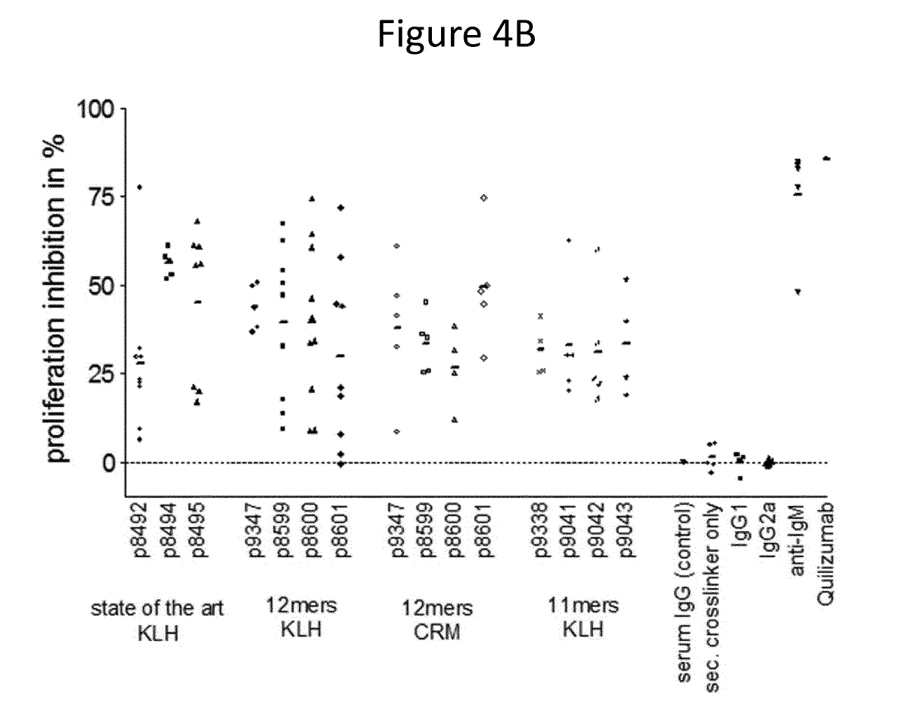

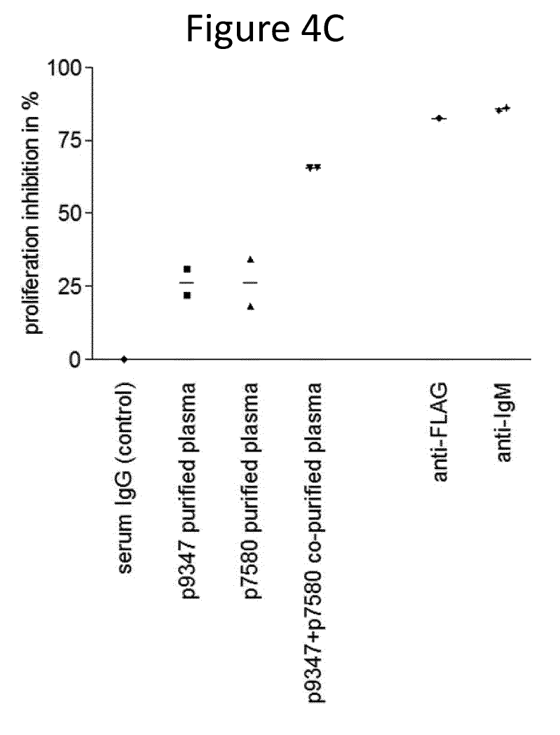

FIG. 4A: Identification of short immunization peptides that induce antibodies able to crosslink the IgE-BCR by specifically binding to EMPD.

FIG. 4B: Identification of vaccine peptides inducing anti-EMPD antibodies with similar IgE-BCR crosslinking activity than prior art immunogens containing medium and large-size fragments of human EMPD.

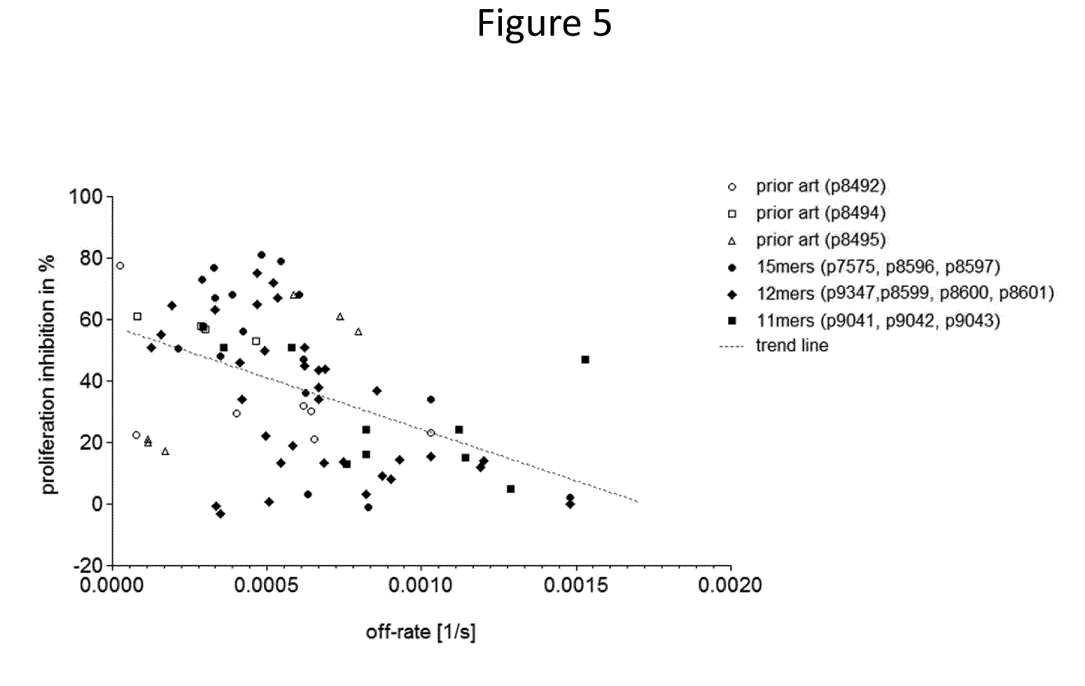

FIG. 5: The off-rate of vaccine-induced antibodies correlates with IgE-BCR crosslinking activity. Short peptides of the present invention (such as p9347, p8599, p8600, p8601, p9041, p9042, p9043) achieve similar binding properties than long and medium size prior art-derived peptides (p8492, p8494 and p8495).

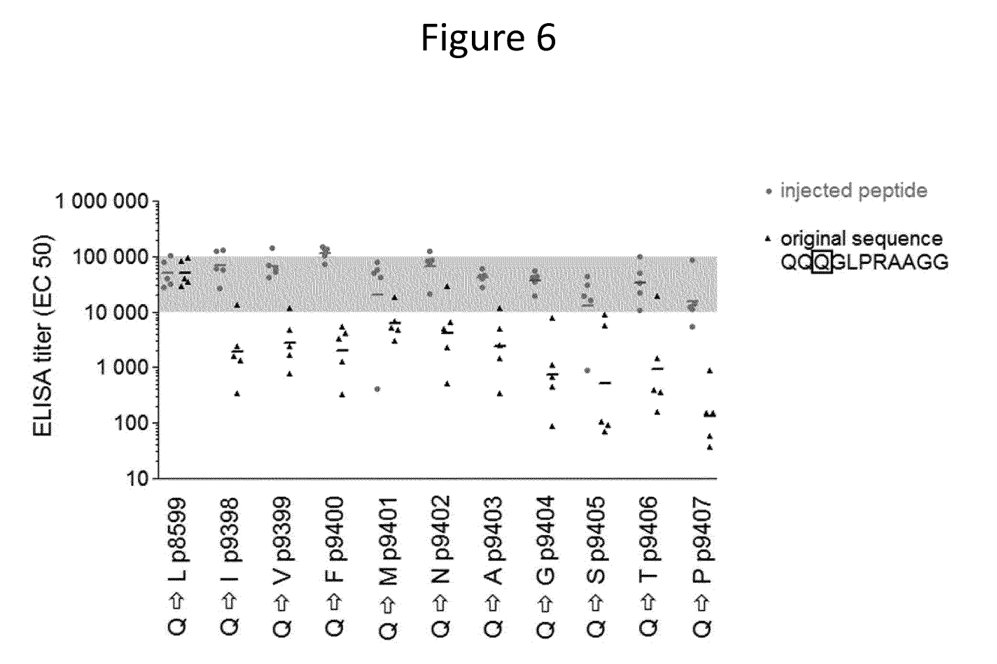

FIG. 6: Variant peptides of p9347 that are immunogenically or functionally equivalent.

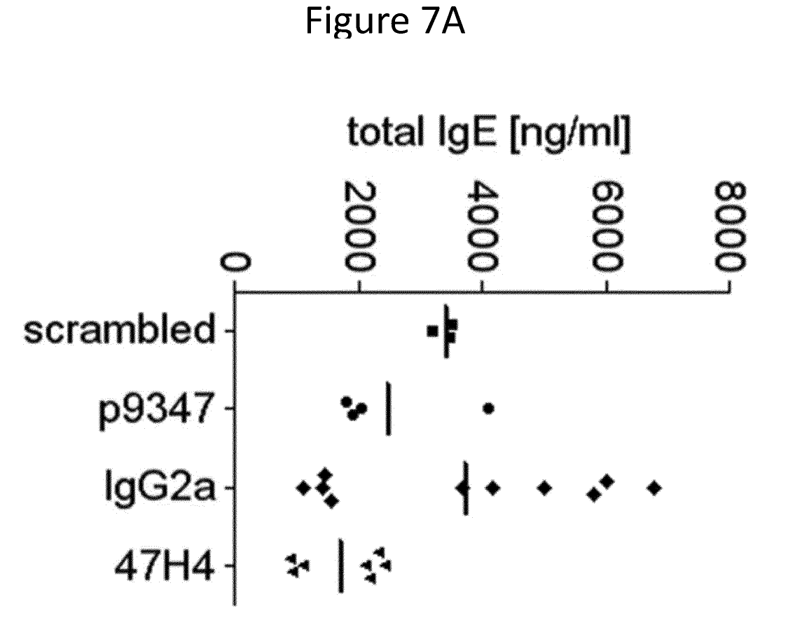

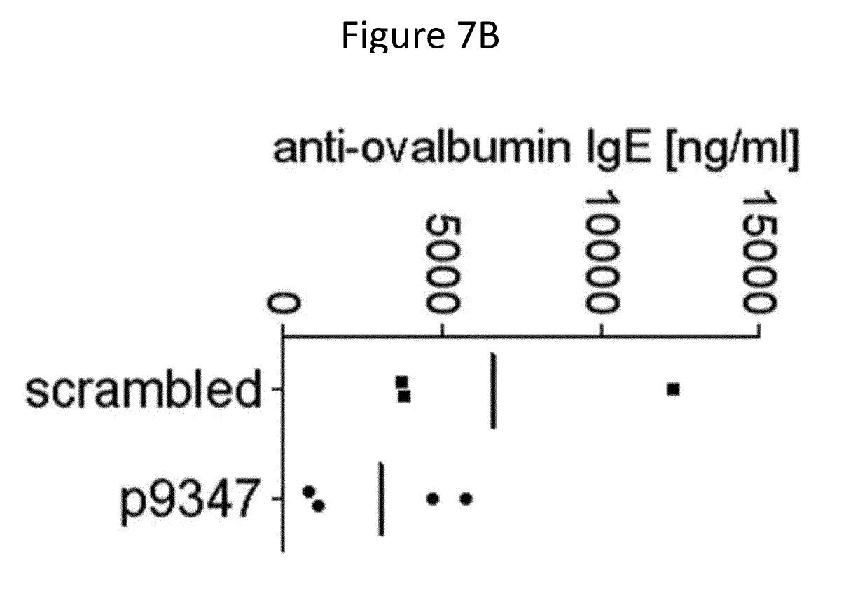

FIG. 7A: Immunizations of transgenic mice with the short peptides of the present invention reduce total IgE levels in vivo.

FIG. 7B: Immunizations of transgenic mice with the short peptides of the present invention reduce ovalbumin specific IgE levels in vivo.

### EXAMPLES

### Example 1: Identification of HLA Class I Binding Peptides Derived from the Human IgE EMPD Region

Several peptides derived from human membrane IgE-EMPD can potentially bind to common HLA class I alleles as predicted by independent HLA binding algorithms (FIG. 1A). This includes also previously published peptides for active anti-IgE-EMPD vaccinations (e.g. the one sequence previously published in pPA-9 from Lin et al. 2012 and US 2014/0220042 A1, and peptide topEMPD-2 from EP 1 972 640 A1). Since it cannot exactly be predicted to what extent these particular peptides will be generated by membrane IgE expressing cells and subsequently presented by HLA class I molecules on the cell surface, they might pose a risk for induction of an undesired T cell response as discussed above. Therefore, six of the thirteen previously published peptides that were predicted to bind HLA Class I (FIG. 1A) were confirmed for binding to HLA molecules in vitro as depicted in FIG. 1B. In contrast, several newly designed peptides of the present invention, including p9347-2 to -4, p8599-2 to -4, p8600-1 and -2 do not bind to HLA class I alleles as listed in FIG. 1B and will therefore not induce an undesired T cell response against membrane IgE-EMPD expressing B cells in these alleles.

HLA class II binding by the short peptides of the present invention is unlikely since 11mers and 12mer are at the lower end of the usual HLA class II binders [Hemmer et al 2000].

FIG. 1A displays prediction scores for 7 relevant HLA class I alleles analyzed by diverse binding prediction algorithms, as indicated by letters S, N and P for SYFPEITHI [Rammensee et al 1999], netMHC [Lundegaard et al 2008], PREDEP (Schueler-Furman et al. 2000] respectively, in order to obtain an improved sensitivity and specificity of the prediction.

This combined judgment, allows a clear distinction of (group 1) best HLA binding candidates derived from the entire EMPD region (top EMPD peptides), (group 2) fragments derived from pPA-9, a human EMPD-derived VLP vaccine containing the pPA-9 sequence by Lin et al 2012 and US 2014/0220042 A1 (prior art I peptides) and (group 3) fragments derived from the p8495 sequence used for the VLP vaccine by Lin et al 2012 and pPA-1 of WO 1996/012740 A1 (prior art II peptides) when compared against vaccine peptides of the present invention (group 4) fragments derived from the claimed peptides of the present invention including p9347, p8599, p8600, p8601, p9338, p9041 and p9042. The top two ranked HLA class I binding scores of each column (according to the indicated prediction methods) are highlighted in gray pointing to the differences between previously proposed active vaccines with long peptides see groups (1)-(3) and the peptides of the present invention with short peptides which show a significantly lower risk (see group (4)). Peptide topEMPD-2 is part of a sequence as claimed by patent EP 1 972 640 A1 (peptide pPA-13).

Binding to HLA class I molecules was compared to a known T cell epitope/a positive reference peptide (defined as 100%). Tested alleles are listed in columns, tested peptides in lines grouped as indicated. Additionally, three peptides derived from p7577, p7580 and p7575 sequences, which were predicted by SYFPEITHI with the highest score, each were tested as pools in vitro in some HLA class I alleles as above. Values above the observed value for a known T cell epitope from human hepatitis C virus (HCV) [Lauer 2004] of 67.5% are considered “binding peptides” and highlighted. Some combinations were not determined and are indicated as “n.d.”

In conclusion, the claimed vaccine peptides of the present invention don't bind to the HLA class I alleles shown in FIG. 1B.

TABLE 1
Integrated peptide and sequence table indicating
origin of peptides, sequences and usage/purpose
of the present patent submission as indicated.

| SEQ | | | | |
|---|---|---|---|---|
| ID | | Peptide | | |
| No. | peptide | name | ation | peptide sequence |
| 1 | | p9347 | | C-QQQGLPRAAGG |
| 2 | E1526 | p8599 | | C-QQLGLPRAAGG |
| 3 | E1527 | p8600 | | C-QQQGLPRAAEG |
| 4 | E1528 | p8601 | | C-QQLGLPRAAEG |
| 5 | — | p9338 | | C-QQQGLPRAAG |
| 6 | E1540 | p9041 | | C-QQLGLPRAAG |
| 7 | E1541 | p9042 | | C-QQQGLPRAAE |
| 8 | E1542 | p9043 | | C-QQLGLPRAAE |
| 9 | | p7575 | | HSGQQQGLPRAAGG-C |
| 10 | E1523 | p8596 | | C-HSGQQLGLPRAAGG |
| 11 | E1524 | p8597 | | C-HSGQQQGLPRAAEG |
| 12 | E1525 | p8598 | | C-HSGQQLGLPRAAEG |
| 13 | | p7580 | | QSQRAPDRVLCHSG |
| 14 | | p7577 | | GSAQSQRAPDRVL-C |
| 15 | | p7572 | | C-GAGRADWPGPPE |
| 16 | | p7593 | | C-AGRADWPGPPELDV |
| 17 | | p7585 | | CggWPGPPELDV |
| 18 | E4802 | p8492 | | C-HSGQQQGLPRAAGGSVPHPR |
| 19 | E4804 | p8494 | | HSGQQQGLPRAAGGSVPHPR-C |
| 20 | E4812 | p8495 | | GLAGGSAQSQRAPDRVLCHSGQQQGLPRAAG |
| | | | | GSVPHPR |
| 21 | pPA-1 | walfield | Seq 1 | GLAGGSAQSQRAPDRVLCHSGQQQGL |
| 22 | pPA-2 | walfield | Seq 2 | PELDVCVEEAEGEAPWT |
| 23 | pPA-3 | e-migis peptide | | ELDVCVEEAEGEAPW |
| 24 | pPA-4 | ARAP3 homology | | TQLLCVEAFEGEEPW |
| 25 | pPA-5 | | | RADWPGPPELDVCVEE |
| 26 | pPA-6 | | | RADWPGPP |
| 27 | pPA-7 | | | SVNPGLAGGSAQSQRAPDRVL |
| 28 | pPA-8 | E4801 | p8491 | SVNPGLAGGSAQSQRAPDRVLC |
| 29 | pPA-9 | | | HSGQQQGLPRAAGGSVPHPR |
| 30 | pPA-10 | E4803 | p8493 | CGAGRADWPGPP |
| 31 | pPA-11 | | | GAGRADWPGPP |
| 32 | pPA-12 | | | GLAGGSAQSQRAPDRVL |
| 33 | pPA-13 | | | GPPELDVCVEEAEGEAP |
| 34 | pPA-I#1 | lin sh 1 | | GLPRAAGGSV |
| 35 | pPA-I#2 | lin sh 2 | | HSGQQQGLPR |
| 36 | pPA-I#3 | lin sh 3 | | PRAAGGSVPH |
| 37 | pPA-I#4 | lin sh 4 | | LPRAAGGSV |
| 38 | pPA-I#5 | lin sh 5 | | RAAGGSVPH |
| 39 | pPA-II#1 | lin lo 1 | | RVLCHSGQQQ |
| 40 | pPA-II#2 | lin lo 2 | | GLAGGSAQS |
| 41 | pPA-II#3 | lin lo 3 | | QRAPDRVLCH |
| 42 | pPA-II#4 | lin lo 4 | | SQRAPDRVL |
| 43 | pPA-II#5 | lin lo 5 | | RAPDRVLCH |
| 44 | pPA-II#6 | lin lo 6 | | QRAPDRVLC |
| 45 | topEMPD-1 | boEMPD-1 | | WPGPPELDV |
| 46 | topEMPD-2 | boEMPD-2 | | GPPELDVCV |
| 47 | topEMPD-1 | boEMPD-1 | | WPGPPELDV |
| 48 | | p9347-2 | | QQQGLPRAA |
| 49 | | p9347-3 | | QQGLPRAAG |
| 50 | | p9347-4 | | QGLPRAAGG |
| 51 | topEMPD-2 | boEMPD-2 | | GPPELDVCV |
| 52 | | p8599-2 | | QQLGLPRAA |
| 53 | | p8599-3 | | QLGLPRAAG |
| 54 | | p8599-4 | | LGLPRAAGG |
| 55 | | p8600-1 | | QQGLPRAAE |
| 56 | | p8600-2 | | QGLPRAAEG |
| 57 | | p9178 | | HSGQQQGLPR |
| 58 | | p9179 | | GLPRAAGGC |
| 59 | | p9180 | | SGQQQGLPR |
| 60 | | p9171 | | SQRAPDRVL |
| 61 | | p9172 | | QRAPDRVL |
| 62 | | p9176 | | QRAPDRVLCH |
| 63 | | p9170 | | QRAPDRVL |
| 64 | | p9171 | | SQRAPDRVL |
| 65 | | p9172 | | QRAPDRVLC |
| 66 | | p7684 | | RAVSVNPGLAGG-C |
| 67 | | p7692 | | AVSVNPGLAGGS-C |
| 68 | | p7693 | | VSVNPGLAGGSA-C |
| 69 | | p7694 | | SVNPGLAGGSAQ-C |
| 70 | | p7695 | | VNPGLAGGSAQS-C |
| 71 | | p7696 | | NPGLAGGSAQSQ-C |
| 72 | | p7578 | | GLAGGSAQSQR-C |
| 73 | | p7569 | | C-GLAGGSAQSQRAPD |
| 74 | | p7583 | | C-GGAQSQRAPDR |
| 75 | | p7582 | | AQSQRAPDR-ggC |
| 76 | | p7581 | | C-SAQSQRAPDRVL |
| 77 | | p7579 | | SAQSQRAPDRVL-C |
| 78 | | p7584 | | Cgg-SQRAPDRVL |
| 79 | | p7576 | | APDRVLCHSGQQQG-C |
| 80 | | p7589 | | RVLCHSGQQQGLPR |
| 81 | | p7590 | | C-QQQGLPRAAGGSVP |
| 82 | | p7574 | | LPRAAGGSVPHPR-C |
| 83 | | p7591 | | AAGGSVPHPRCHAG |
| 84 | | p7573 | | C-VPHPRAHAGAGRA |
| 85 | | p7592 | | HPRAHCGAGRADWP |
| 86 | | p7586 | | WPGPPELDV-ggC |
| 87 | | p7571 | | DWPGPPELDVCVEE |
| 88 | | p7594 | | PPELDVCVEEAEG |
| 89 | | p7588 | | Cgg-LDVAVEEAEG |
| 90 | | p7587 | | DVAVEEAEGEA-ggC |
| 91 | | p7570 | | LDVCVEEAEGEAPW |
| 92 | | p7595 | | CVEEAEGEAPW |
| 93 | E1517 | p8591 | | HSGQQLGLPRAAG-C |
| 94 | | p9437 | | (biotin-Aca-Aca)C-QQQGLPRAAGG |
| 95 | E07/15bio | p9195 | | HSGQQQGLPRAAGG-C K (biotin-Aca) |
| 96 | | p9267 | | AVSVNPGLAGGSAQSQRAPDRVLCHSGQQQG |
| | | | | LPRAAGGSVPHPRCHCGAGRADWPGPPELDV |
| | | | | CVEE-K(Biotin-Aca) |
| 97 | | p9457 | | CHSGQQQGLPRAAGGSVPHPRCH-K- |
| | | | | (biotin-Aca) |
| 98 | | p9458 | | CHSGQQQGLPRAAGGSVPHPRCH-K- |
| | | | | (biotin-Aca) with C-C bridge |
| 99 | | p9398 | | C-QQIGLPRAAGG |
| 100 | | p9399 | | C-QQVGLPRAAGG |
| 101 | | p9400 | | C-QQFGLPRAAGG |
| 102 | | p9401 | | C-QQMGLPRAAGG |
| 103 | | p9402 | | C-QQNGLPRAAGG |
| 104 | | p9403 | | C-QQAGLPRAAGG |
| 105 | | p9404 | | C-QQGGLPRAAGG |
| 106 | | p9405 | | C-QQSGLPRAAGG |
| 107 | | p9406 | | C-QQTGLPRAAGG |
| 108 | | p9407 | | C-QQPGLPRAAGG |
| 109 | | p9347 | | QQQGLPRAAGG |
| 110 | E1526 | p8599 | | QQLGLPRAAGG |
| 111 | E1527 | p8600 | | QQQGLPRAAEG |
| 112 | E1528 | p8601 | | QQLGLPRAAEG |
| 113 | — | p9338 | | QQQGLPRAAG |
| 114 | E1540 | p9041 | | QQLGLPRAAG |
| 115 | E1541 | p9042 | | QQQGLPRAAE |
| 116 | E1542 | p9043 | | QQLGLPRAAE |
| 117 | | p7575 | | HSGQQQGLPRAAGG |
| 118 | E1523 | p8596 | | HSGQQLGLPRAAGG |
| 119 | E1524 | p8597 | | HSGQQQGLPRAAEG |
| 120 | E1525 | p8598 | | HSGQQLGLPRAAEG |
| 121 | | p7580 | | QSQRAPDRVLCHSG |
| 122 | | p7577 | | GSAQSQRAPDRVL |
| 123 | | p7572 | | GAGRADWPGPPE |
| 124 | | p7593 | | AGRADWPGPPELDV |
| 125 | | p7585 | | WPGPPELDV |

“C-” followed or “-C” preceded by the sequence indicates that the cysteine needed to attach the peptide to the carrier is not part of the original protein-sequence, while “C” followed preceded by the sequence indicates a naturally occurring Cysteine (the same applies for a Glycine-Glycine-Cysteine linker (“-ggC”, “Cgg-”) or other linkers); peptide names (“pXXXX”) for the C-coupled peptide and the peptide without added C are the same due to the identical core sequence.

### Example 2: Immunogenicity and Target Accessibility of Peptide Vaccine-Induced Immune Sera

Peptides p7577, p7580 and p7575 provide the highest MFI ratios on Ramos cells although their titers are the same (or lower) than the one of other peptides as shown in FIG. 2A. Unexpectedly, peptides p7577, p7580 and p7575 and the derivatives of the later (p9347, p8599, p8600, p8601) are therefore the most suitable candidates for a carrier protein-based peptide vaccine.

Mouse plasma, taken after 4 biweekly injections of an anti-human EMPD peptide vaccine (composed of peptide-carrier conjugate with KLH or CRM mixed with Alum as adjuvant) were tested by standard ELISA procedure for determining titers against the injected peptide coupled to BSA. Titers were calculated by EC50 of their dilution using a four-parameter curve fitting and show mostly values between 10̂4 and 10̂5 (gray interval on the y-axis). Each dot represents the titer of one animal, the horizontal line shows the geometric mean from each animal group immunized with the peptide indicated on the x-axis. Together, all tested peptides that are covering the entire human EMPD sequence, as well as single and double amino acid exchanges (p8599, p8600, p8601) are immunogenic in mice and can therefore be regarded as possible immunogens for active anti-EMPD vaccinations. As shown in FIG. 2A, all injected peptides are immunogenic.

The same immune sera as in FIG. 2A were used for affinity purification of polyclonal antibodies using the same peptide as used for immunization (peptides as indicated in FIG. 2A) to allow a titer-independent staining on HEK wt (background signal) or HEK-C2C4 (specific signal) expressing cells. From the staining intensities (MFI) of these populations, a specificity index (SI) was calculated according to the formula described under materials and methods and plotted on the y-axis. Higher SI's reflect higher specificity of target binding (such as positive control mABs anti-IgE Le27 and BSW17 on the right side), while a SI around 1 indicates that HEK-wt and HEK-C2C4 cells are recognized equally well indicating the absence of specific target interaction (depicted as “specificity threshold” on the y-axis), such as e.g. mouse IgG controls, the third, fourth and fifth sample from the right. HEK wt cells showing a strong background signal were given a SI value of 0.2. Each dot represents affinity purified antibodies from one animal or control ABs, the horizontal line shows the mean for each group immunized with the peptide as indicated on the x-axis. Remarkably, although all injected peptides are similarly immunogenic (FIG. 2A), the accessibility of the different stretches of EMPD in a cellular context is restricted to only a few regions such as e.g. p7580 and p7575 or p7572, p7593 and p7585 (FIG. 2B). This unpredictable characteristic was further confirmed in a cellular model expressing a surrogate for the “natural” form of IgE EMPD, namely in presence of Ig-alpha and Ig-beta chains as shown in FIG. 2C.

The same samples as in FIG. 2B were used for staining membrane IgE C2C4-negative or membrane IgE C2C4-positive Ramos cells for EMPD using a given affinity purified antibody concentration (25 ug/ml) in a titer-independent manner. The ratio of staining intensities on the y-axis is calculated by the staining intensity (MFI) on membrane IgE C2C4-expressing cells divided by the membrane IgE-C2C4 negative background signal from non-induced cells. A MFI ratio around or below 1 (labelled “specificity threshold”, on the y-axis [dotted line]) reflects no specific staining of the target. Negative controls (right sample block, starting with “no primary AB”) and positive controls (right sample block, starting with “anti-IgE (Le27)”) show MFI ratios around 1 or above 5, respectively. MFI ratios higher than 1 indicate a specific cell surface signal (such as e.g. positive control mABs anti-IgE Le27 and BSW17; right side of the panel).

Since Ramos cells, unlike HEK cells, express endogenous BCR associated with Ig alpha and Ig beta, they reflect the accessibility of certain EMPD epitopes in a more natural structural context than without Ig-alpha and -beta. The region covered by peptides p7572, p7593 and p7585 was previously described by Chen et al, 2010 to be shielded or negatively influenced by the expression of Ig alpha and Ig beta and is therefore not recognized on Ramos cells in contrast to the signal on HEK cells that do not express these accessory proteins. Each dot represents one animal, the line shows the mean for each group immunized with the peptide as indicated on the x-axis (in case of control ABs each symbol represents an independent biological replicate).

### Example 3: Claimed Peptides of the Present Invention Lack Induction of Off-Target Binding Immune Sera to Human PBMCs

Off-target binding to a widely expressed protein (ARAP3, pPA-3) has been observed by mABs targeting a region of human EMPD in the region of p7570 (FIG. 2) or pPA-4 (Chowdhuy et al, 2012). It is therefore necessary to assess the present vaccine peptides for their risk of inducing an off-target immune response similar to these mABs.

The same immune sera and antibody purifications of KLH/peptide vaccine immunized mice are the same as in FIGS. 2B and 2C. They were tested for undesired, off-target binding to cell surface antigens. As a surrogate for easily accessible, plasma-exposed human cells, PBMCs derived from two healthy donors were used for flow cytometric staining (PBMC binding [MFI] shown on the y-axis). Since IgE-BCR-positive B cells are barely detectable in peripheral blood, they fall below conventional FACS detection limits in such analyses [Davies et al 2013]. As shown in the central three groups of samples (available immune sera as indicated on the x-axis), PBMC-binding signals from all tested p7575-derived immune sera remained within background levels, whereas large peptide-derived immune sera (see left block “p8492, p8494, p8495”) yielded clear positive signals reflecting unspecific off-target binding to undefined cell surface antigens. Each group of four bars represents off-target measurement with one plasma sample against B cells and non-B-cells from PBMCs of three healthy donors, respectively, as indicated by the differently shaded bars within the panel. Light grey bars reflect unspecific binding to B220 positive B cells, dark grey bars reflect off-target binding to B220 negative cells (i.e. non-B cells within PBMCs). Isotype controls and an anti-human HLA-DR used a positive staining control is shown on the right.

As shown in FIG. 3, the peptides of the present invention induce IgE EMPD-specific antibodies that, in contrast to previously proposed active vaccines (such as those proposed by Lin et al 2012 or US 2014/0220042 A1), do not show unspecific off-target binding to human PBMCs.

### Example 4: IgE-BCR Crosslinking Activity of Claimed Vaccine Peptides

The same antibodies, immune sera and affinity purifications as in FIGS. 2B and 2C were preselected for their IgE EMPD-specificity and for their ability to crosslink the IgE-BCR. As surrogate for functional IgE-BCR crosslinking by antibodies, membrane IgE C2C4-expressing Ramos cells (as in example 2C) were incubated with test or control antibody and measured for functional proliferation inhibition as measured by relative EdU incorporation (plotted on the y-axis) against control IgG (set to 100%). As shown on the right side of the panel, anti-IgM binding to the endogenously expressed BCR of Ramos cells is used as a positive control for proliferation inhibition by BCR crosslinking. Each dot represents relative proliferation inhibition activity (in %) of affinity purified anti-EMPD or control antibodies derived from one animal (in case of anti-IgM each symbol represents an independent biological replicate). The horizontal line depicts the mean crosslinking activity from each vaccinated animal group as indicated by the respective peptide name on the x-axis. In conclusion, it was found that peptide p7575 had strongest crosslinking activity when compared to other EMPD vaccine peptides.

In order to provide vaccine peptides that are devoid of any T cell epitope, it is necessary to use short peptides (e.g. in the range of <12-15 AA) instead of long peptides (e.g. >20AA) that might contain HLA class I and/or -class II binding T cell epitopes. However at the same time it is not evident whether shortening of immunization peptides will yield antibody responses that maintain efficient IgE-BCR crosslinking activity. For this purpose in FIG. 4B, short peptide-induced immune sera as in FIGS. 2B, 2C and 3B were screened for their ability to crosslink IgE-BCR (as demonstrated in IgE C2C4 expressing Ramos cells). As a surrogate for functionality readout, the relative proliferation inhibition activity is expressed as shown in FIG. 4A and plotted on the y-axis. Quilizumab, a humanized mAB recognizing and crosslinking human EMPD, was used as additional positive control. Unexpectedly, short 11mer (p9338, p9041, p9042, p9043) and 12mer peptides p9347, p8599, p8600, p8601) from the present invention induce immune sera that yield comparable crosslinking activity than previously published large peptides not suited for vaccination because of their T cell epitopes (as exemplified by prior art-derived peptides p8492, p8494 and p8495). The short peptides of the present invention therefore contain sufficient epitope information to allow for the induction of IgE-BCR-crosslinking antibodies despite their reduced size. Symbols, peptides and controls are indicated on the x-axis as in FIG. 4A.

In order to test synergistic effects upon vaccination with multiple EMPD peptides in FIG. 4C rabbits were injected simultaneously with p9347 and p7580 on opposite flanks. Antibodies were purified and tested for crosslinking activities as in FIGS. 4A and 4B. As surrogate for functional IgE BCR crosslinking by the induced antibodies, membrane IgE C2C4-expressing Ramos cells (as in example 4A and 4B) were incubated with test or control antibody and measured for functional proliferation inhibition as measured by relative EdU incorporation (plotted on the y-axis) against control serum IgG (set to 100%). As expected antibodies directed against a single epitope showed intermediate crosslinking activity, while their combination lead to an unexpected synergistic effect (at the same total concentration as the single epitopes). Anti-IgM (binding to the endogenously expressed BCR of Ramos cells) and anti-FLAG (binding to the FLAG tag on the induced IgE C2C4 protein) antibodies were used as positive controls. Symbols, peptides and controls are indicated on the x-axis as in FIG. 4A.

In conclusion, it was found that by combining the antibodies induced in one animal by immunising against two different regions of EMPD the resulting crosslinking effect synergizes to a stronger proliferation inhibition than the single epitopes alone.

FIG. 4A summarizes the identification of short immunization peptides that induce antibodies able to crosslink the IgE-BCR by specifically binding to EMPD; FIG. 4B shows the identification of vaccine peptides inducing anti-EMPD antibodies with similar IgE-BCR crosslinking activity than prior art immunogens containing medium and large-size fragments of human EMPD. FIG. 4C shows the synergistic effect upon combination of different epitope for vaccination.

### Example 5: Correlation Between Crosslinking Activity and Affinity to Human EMPD

KLH-peptide vaccine induced immune sera (as in FIGS. 2, 4A and 4B) were analyzed by surface plasmon resonance for their off-rates to peptide (p9267) covering the entire human EMPD region with exception of the 5 C-terminal amino acids. The calculated off-rate (in 1/s; indicated on the x-axis) defines one parameter of the affinity. Functional IgE-BCR crosslinking in Ramos cells (as reflected by proliferation inhibition activity as in FIG. 4) is plotted on the y-axis. In conclusion, short vaccine peptides such as most preferably p9347(*), p8599, p8600, p8601, p9338(*), p9041, p9042, p9043 but also p7575, p8596, p8597 according to the present invention, induce antibodies that show good correlation of their off-rates and functional IgE-BCR crosslinking activity (Pearson r=−0.4725; p value (two-tailed)<0.0001; R2=0.2232).

FIG. 5 shows that the off-rate of vaccine-induced antibodies correlates with IgE-BCR crosslinking activity. Short peptides of the present invention (such as p9347, p8599, p8600, p8601, p9338, p9041, p9042, p9043) achieve similar binding properties than long and medium size prior art-derived peptides (p8492, p8494 and p8495).

### Example 6: Modifications of Claimed Peptides

Mice were immunized as in Example 6 with peptides p8599, and similar peptides containing single amino acid exchanges at a same defined position (boxed as indicated originally a “Q”). Exchanges were placed based on physico-chemical properties of the amino acid. In order compare the immunogenicity of the individual variants, immune sera were analyzed by ELISA for their titer (EC50) against the injected peptide (grey dots) and plotted on the y-axis. The cross-reactivity (EC50) of the induced immune sera to the original peptide is plotted with filled triangles. Each symbol represents the titer against the original sequence of p9347 or the injected peptide from one animal, the horizontal line shows the geometric mean from each animal group immunized with the peptide with the respective exchange indicated on the x-axis.

Unexpectedly, amino acid substitutions as indicated on the x-axis (*) keep or even improve the immune response that can be achieved by the original sequence (p9347) in a manner that was unpredictable by physicochemical or any other parameters. Similarly, binding and crosslinking data with peptide p8600 and p8601 (Examples 2, 4, 5 and 6) demonstrate that it is as well possible to substitute the second last position of p9347 from G to E thereby maintaining full functionality also in double substitutions such as shown for p8601.

### Example 7: Demonstration of In Vivo IgE Suppression in Animal Model

Passive administration of affinity purified antiserum obtained from p9347-vaccine immunized mice (as in FIG. 2) suppresses total IgE and Ovalbumin(Ova)-specific IgE as shown in FIGS. 8A and B, respectively. In order to induce IgE, mice were treated with Ova (Sigma) three times (days 2, 15 and 23 of the vaccination protocol). Plasma was taken at day 27 and total and Ova-specific mouse IgE was quantified by ELISA (Biolegend and Cayman Chemical, respectively) as indicated on the y-axis. In vivo functional activity of antibodies was tested by weekly passive transfer into a newly created homozygous IgE-huEMPD knock-in mouse model where the endogenous mouse IgE-EMPD encoding exon had been replaced by the homologuous human sequence (long variant; SEQ ID NO: 126 to be assigned: GLAGGSAQSQRAPDRVLCHSGQQQGLPRAAGGSVPHPRCHCGAGRADWPGPPELDVCVEEAEGE A) using a Znf strategy in a Balb/c background. A scrambled control peptide (designated “scrambled”; p9553: CLAGQGRQPQGA; SEQ ID NO: 127 to be assigned) and monoclonal control antibodies mAB IgG2a (isotype control; Biolegend) and mAB 47H4 as a positive reference (EP2132230B1, U.S. Pat. No. 8,632,775B2 and US20090010924; mouse ancestor of Quilizumab®) were used for control purposes. Each dot represents the IgE level from one animal. The horizontal line depicts the mean IgE levels from each vaccinated animal group as indicated by the respective peptide name (or mAB) on the x-axis. In conclusion, passive transfer of p9347-specific antisera reduces total IgE (FIG. 8A) and Ova-specific IgE (FIG. 8B). These data provide an example for how antibodies that are induced by a peptide p9347-based vaccine according to the present invention can inhibit total IgE and suppress Ova-induced IgE in vivo as a surrogate for allergen-specific IgE.

### Material and Methods

### Example 1—Material & Methods

### FIG. 1A:

In order to obtain reasonable HLA binding prediction sensitivity, 2 or 3 most distinct MHC binding prediction methods were applied using three online prediction programs (SYFPEITHI [http://www.syfpeithi.de]; netMHC [http://www.cbs.dtu.dk/services/NetMHC/]; PREDEP [http://margalit.huji.ac.il/Teppred/mhc-bind/index.html]), which are based on different algorithms including motif matrices, ANN-regression and threading, respectively. This allowed for the identification of potential common HLA-A and -B binding 9-mer peptides derived from vaccine peptides as indicated in FIG. 1A. In order to provide a sensitive strategy, for HLA binder identification, peptides with the highest predictions in any of the programs were analyzed by the remaining program(s) as well. SYFPEITHI predictions are given as score reaching from 0 (no binding) to 36 (maximum binding). netMHC estimates the affinity (in nM), where 0 to 50 nM are considered strong binders and weak binder threshold score is 500 nM. PREDEP calculates an “energy score” (lowest value=maximum binding). For some of the alleles tested, PREDEP cannot predict binding for the given peptide length and is therefore used at the next shorter peptide length.

### FIG. 1B:

For biochemical confirmation of HLA binding, an in vitro binding assay was applied. The high-throughput ProImmune REVEAL® binding assay determines the ability of each candidate peptide to bind to one or more HLA class I alleles and stabilize the HLA-peptide complex. [Schwabe et al 2008]. By comparing the binding of a test peptide with binding of a high affinity reference T cell epitope, the most likely immunogenic peptides in a protein sequence can be identified. Detection is based on the presence or absence of the native conformation of the MHC-peptide complex. Candidate peptides from FIG. 1A were assembled, according to the project specifications, with the alleles indicated in FIG. 1A and analyzed using the ProImmune REVEAL® MHC-peptide binding assay to determine their level of incorporation into MHC molecules. Binding to MHC molecules was compared to that of a known T cell epitope, a positive control peptide, with very strong binding properties. The ProImmune REVEAL® binding score for each MHC-peptide complex is calculated by comparison to the binding of the relevant positive control. Peptides that may be immunologically significant or warrant further investigation as good binders are considered to be those peptides with scores equal or higher than that of a known T cell epitope (HCV E1 207-214 was used) [Lauer 2004)]. Experimental standard error was obtained by triplicate positive control binding experiments. The standard error for this control is reported below as an illustration of the degree of error that can be obtained in a ProImmune REVEAL® MHC-peptide Binding Assay.

In a second set of experiments pools of equimolar mixtures of the three given peptides were tested for binding on certain alleles from FIG. 1 as indicated and additionally on A*01:01, A*24:02, A*29:02, B*08:01, B*14:01, B*40:01.

### Example 2—Material & Methods

The ELISA protocol was performed in 96-well Nunc MaxiSorp plates which were coated with 10 mM of the appropriate peptide-BSA conjugate (Bovine BSA Sigma with GMBS Applichem), diluted in PBS, followed by blocking with 1% BSA in PBS, for 1 h at room temperature while shaking overnight at 4° C. Plasma dilutions were added to the wells, serially diluted in 1×PBS, 0.1% BSA, 0.1% Tween-20 and incubated while shaking for 1 h at RT, followed by 3 washes with 1×PBS 0.1% Tween-20. For detection, biotinylated anti-mouse IgG1 (H+L) (Southern Biotech. dilution 1:2000) was added for 1 h at RT while shaking, washed 3 times with 1×PBS 0.1% Tween-20, followed by horseradish peroxidase coupled to streptavidin (Roche, 0.1 U/ml) for 30 min at 37° C. For visualization, the substrate ABTS (BioChemica, AppliChem) was added after 3 washes with 1×PBS 0.1% Tween-20. After 30 min incubation at RT while shaking, the reaction was stopped with 1% SDS. The optical density was measured at 405 nm with a microwell plate reader (Sunrise, Tecan, Switzerland). Graphpad (Prism) was used to calculate the EC50, called peptide titer, by non-linear regression analysis with four parameter curve fitting.

### Vaccination Protocol:

Peptides were synthesized by FMOC solid phase peptide synthesis (EMC microcollections GmbH, >95% purity), some with additional N or C terminal cysteins for coupling (when necessary). The peptide was coupled to the carrier protein Keyhole Limpet Hemocyanin (KLH, Biosyn GmbH or Sigma Aldrich) or to C-reactive recombinant CRM197 diphtheria toxin mutant protein (CRM pre-clinical grade, PFEnex, San Diego) using N-gamma-Maleimidobutyryl-oxysuccinimide ester (GMBS, Applichem). Peptide-carrier conjugates were adsorbed to aluminum hydroxide (Alum, Brenntag) as adjuvant. The vaccine dose contained 30 μg peptide plus 0.1% Alum. Female wild-type Balb/c (Janvier, St. Berthevin) aged 8-12 weeks were injected subcutaneously (s.c.) into the flank four times at biweekly intervals. Plasma was taken two weeks after the last injection.

### Membrane IgE C2C4 Human EMPD Cell Model:

Human Burkitt's lymphoma-derived Ramos cells (Ramos-ERHB, ECACC no 85030804) were cultured in RPMI-1640 medium, 10% FCS, antibiotics at 5% CO2/37° C. TET-inducible expression of membrane IgE-C2C4 containing an N-terminal FLAG-tag followed by the IgE heavy constant chain (domains 2-4, followed by human EMPD, TM and IC region of the human IgE-BCR was constructed by gene synthesis, cloned into a TET-inducible expression vector, and stably transfected into Ramos cells together with the appropriate regulator construct. The resulting cell line expresses an inducible IgE-BCR model and providing a model for natural human EMPD exposure on the cell surface in the presence of Ig-alpha and -beta allowing for assessment membrane IgE crosslinking and cellular signaling. Membrane IgE C2C4 expression is induced by addition of 500 ug/ml Doxycyclin (Clontech) overnight, designated “C2C4” throughout the text. In contrast, non-induced cells (designated “wt”) don't express membrane IgE C2C4. Furthermore, HEK Freestyle cells (FreeStyle™ 293-F Cells, Invitrogen) were cultured in shaking Erlenmeyer Freestyle medium (Gibco) at 37° C. (called “wt”). A stable HEK-Freestyle membrane IgE-C2C4 expressing cell clone was generated using a CMV-driven mammalian expression vector driving the same construct than in the inducible Ramos cells.

Affinity Purification of Polyclonal ABs from Plasma:

For staining and crosslinking experiments, peptide vaccine-induced antibodies were affinity purified from mouse/rabbit plasma by coupling the injected peptide to magnetic beads via Cystein (1 □m BcMag iodoacetyl activated, Bioclone) according to the manufacturer's guidelines followed by incubation of 50 μl mouse plasma for 2 h at RT under constant agitation. After binding, beads were washed 8 times and subsequently eluted using 0.2 M glycine, 0.15 M NaCl at pH 1.9 followed by neutralization with 1M HEPES, pH7.9. Finally, eluted antibodies were concentrated and re-buffered into PBS using Spin-Xr UF500 (Millipore) columns and stored at 4° C. Protein content was quantified by Nanodrop ND-1000 (Thermo Scientific).

### Cell Staining for Flow Cytometry and Determination of the “Specificity Index” and MFI Ratios:

HEK-Freestyle wt and -membrane IgE-C2C4 cells were stained with 25 ug/ml affinity purified antibodies, washed in FACS buffer and incubated with Goat-a-mouse IgG-Biotin (1:500, Southern Biotech) and Strep-PE (1:40, RDSystems). C2C4 cells were stained simultaneously with rabbit a-FLAG (Sigma 9 ug/ml) and PerCP goat anti-rabbit F(ab′)2 (2.5 μg/ml, Jackson Immuno Research).

### Determination of the Specificity Index (SI):

(1) all samples except control non-binders were normalized to the mean PerCP signal, i.e. expression of membrane IgE construct. (2) PE values of both subpopulations were normalized to the PE intensities of mouse IgG1 isotype control. (3) If wt cells had a value of 2 or higher (high binding to wt cells) the SI value was set to 0.2. (4) For all other samples, the SI is obtained by dividing the normalized PE value for C2C4 positive cells by the background value obtained from wt cells.

Ramos (−wt and −C2C4 expressing) cells were stained with vaccine-induced affinity-purified antibodies or control ABs at 25 ug/ml, washed in FACS buffer (PBS 1% FCS) and incubated with AlexaFluor 488 goat-anti-mouse IgG F(ab′)2 (3 μg/ml, Jackson Immuno Research). C2C4 cells were stained simultaneously with rabbit a-FLAG (Sigma 9 ug/ml) and PerCP goat anti-rabbit F(ab′)2 (2.5 μg/ml, Jackson Immuno Research). Cells were acquired on a FACScan (BD) and evaluated in FlowJo (Treestar) acquiring MFI of live wt, FLAG negative cells and live C2C4, FLAG positive populations allowing for determination of the MFI ratio [MFI (membrane IgE-C2C4 positive cells)/MFI (C2C4 negative cells)].

### Example 3—Material & Methods

Plasma from vaccinated mice was used for affinity purification of polyclonal antibodies as described in Example 2.

### Flow Cytometric Analysis of PBMC:

PBMCs from a Buffy coat of healthy donors were purified (Ficoll gradient) and frozen in liquid nitrogen. Cells were taken in culture overnight in RPMI-1640 medium with 10% FCS (both Gibco) and antibiotic and incubated with vaccine induced affinity purified antibodies from mouse- or control ABs at 25 ug/ml (mouse IgG1, from Biolegend and Biogenes, IgG2a and anti-HLA-DR, both form Biolegend at 0.04 ug/ml as technical control), washed in FACS buffer (PBS 1% FCS) and incubated with PE Donkey a-mouse IgG (Fab′)2 (2.5 ug/ml, Jackson Immuno Research). B cells were stained in additional with FITC a-mouse/human CD45R/B220 (10 ug/ml, Biolegend) or Isotype control. Cells were acquired on a FACScan (BD) and evaluated in FlowJo (Treestar) by assessing the MFI of live lymphocytes subpopulations (B cells: CD45R/B220 positive, non-B cells: CD45R/B220 negative).

### Example 4—Material & Methods

### Membrane IgE-Crosslinking Assay:

Ramos cells (wt and C2C4; see example 2) were seeded half a million per sample and incubated with 10 μg/ml of vaccine induced affinity purified or control antibodies as in example 2 in complete medium for 1 h. Cells were spun and resuspended in complete medium (for C2C4 cells with Doxycyclin) with secondary crosslinker goat anti-mouse or anti-rabbit IgG, Fcγ fragment specific, F(ab′)2 fragments from affinity purified antibodies (Jackson Immuno Research) at the same concentration and incubated overnight to induce BCR crosslinking. Quilizumab, a prototypic, humanized monoclonal AB binding human EMPD (Brightbill et al, 2010) was expressed in CHO cells for experimental purpose as re-engineered mouse/human chimaeric AB with a mouse IgG2a constant heavy chain, purified by protein A and used as a positive inhibition control at 1 ug/ml. Goat anti-IgM (Southern Biotech) and rabbit anti-FLAG (Sigma) were used at 3 and 10 ug/ml, respectively, as positive controls.

Two White New Zealand rabbits were immunized on opposite flanks with CRM-p9347 (30 ug) and KLH-p7580 (100 ug) as described for mice in Example 2.

Proliferation was quantified by Click-iT® EdU Alexa Fluor® 488 Flow Cytometry Assay Kit (Invitrogen) according to the manufacturer's instructions. Briefly, 10 μM EdU was added for 1 h before fixation and development. Samples were acquired on a FACScan (BD) and evaluated in FlowJo (Treestar) by assessing the % EdU positive cells. Proliferation inhibition as a surrogate for crosslinking activity was calculated by setting the proportion of EdU positive cells from IgG from plasma (normally around 40%) as 100%.

### Example 5—Material & Methods

### Affinity Determination by BiaCore:

Off-rate of vaccine-induced antibodies was analyzed by surface plasmon resonance (SPR) (BiaCore®) using a Biacore 2000 instrument (GE Healthcare). Biotin-tagged antigen p9267 (EMC, Tubingen, Germany) was immobilized on the surface of a streptavidin-coated BiaCore®-sensor chip using HEPES-buffered saline, pH 7.4 (HBS) as running buffer. A minimum of 50 response units (RU) of the peptide were loaded on the chip, flow cell 1 was left empty and used as a reference (background signal). Subsequently, free streptavidin binding sites were blocked with free biotin (Sigma-Aldrich) and naïve plasma (1:100). 100 μl of each unpurified plasma sample (dilution 1:100 in HBS) at a flow rate of 30 μl/min were injected and the chip surface was regenerated with 15 μl of 10 mM glycine, pH<=2.2 after each plasma injection. After each run, the background signal of the first flow cell was subtracted from the signals obtained by the following, ligand-bound flow cells. The stability of the chip-surface was controlled by repeated injections of control antibody. For evaluation RU values at the end of plasma injection were used as an indicator for the total amount of bound antibody. Off-rate values (1/s) were calculated using the BIA evaluation software (1:1 Langmuir interaction model for dissociation). The off-rate describes the dissociation velocity of the antibodies from the ligand and constitutes, and thereby reflects (beside the on-rate) an important parameter for affinity determination derived from individual plasma samples. Consistently, lower antibody off-rates to human EMPD peptide correlate with relatively stronger IgE-BCR crosslinking activity in the cellular readout system.

Membrane IgE-crosslinking assay: as in Example 4.

### Example 6—Material & Methods

Single amino acid exchanges starting from the original EMPD sequence were chosen based on similar or dissimilar physico-chemical properties. Mice were vaccinates as described under example 2. Immune sera were analyzed on the injected and original peptide as in FIG. 2A.

### Example 7—Material & Methods

Homozygous mice for the human IgE-EMPD were immunized passively by administration of sera from mice injected with the indicated peptide on a carrier protein purified by affinity for the injected peptide or monoclonal antibodies (47H4 or isotype control) at weekly intervals.

Additionally groups were injected with ovalbumin (Sigma) on day 2, 15 and 23. Plasma was taken on day 27 and analyzed for total and ova specific IgE content by ELISA (Biolegend and Cayman Chemical, respectively).

### REFERENCES

Achatz et al., Open Immun J 2008, 1: 25-32 DOI: 10.2174/1874226200801010025]
Ahmed S S et al, Science Translational Medicine 2015: Vol. 7,
Issue 294, pp. 294ra105 DOI: 10.1126/scitranslmed.aab2354
Batista et al., J Exp Med. 1996 Dec. 1; 184(6):2197-205. PMID: 8976175
Benhamou et al., Eur J Immunol. 1990 June; 20(6):1405-7. PMID: 2369921
EP 1 972 640 A1
Brightbill et al., J Clin Invest. 2010 June; 120(6):2218-29. doi: 10.1172/JCI40141. Epub 2010 May 10. PMID: 20458139
U.S. Pat. No. 5,274,075 A
US 2014/0220042 A1
Chang et al., Biotechnology (N Y). 1990 February; 8 (2):122-6. PMID: 1369991
WO 1998/053843 A1
WO 2007127221 A3
Chen et al., Int Arch Allergy Immunol. 2002 August; 128(4):315-24. PMID: 12218370
Chen et al., J Immunol. 2010 Feb. 15; 184(4):1748-56. doi: 10.4049/jimmunol.0902437. Epub 2010 Jan. 18. PMID: 20083663
Chowdhury et al., Mol Immunol. 2012 October; 52(3-4):279-88. doi: 10.1016/j.molimm.2012.06.004. Epub 2012 Jun. 29. Erratum in: Mol Immunol. 2013 January; 53(1-2):172. PMID: 22750228
Davies et al, JACI 2013, Vol. 131 (4): 972-976
Deehan et al., Autoimmun Rev. 2015 Mar. 2. pii: S1568-9972(15)00051-8. doi: 10.1016/j.autrev.2015.02.007. [Epub ahead of print] Review. PMID: 25742758
Feichtner et al., J Immunol. 2008 Apr. 15; 180(8):5499-505. PMID: 18390733
Gauvreau et al., Sci Transl Med. 2014 Jul. 2; 6(243):243ra85. doi: 10.1126/scitranslmed.3008961. PMID: 24990880
Geurts et al., ZFN_Mediated_RatKO, Clin Sc, 2010
Haba et al., Proc Natl Acad Sci USA. 1990 May; 87(9):3363-7. PMID: 2185467
Hemmer et al., Int Immunol. 2000 March; 12(3):375-83. PMID: 10700472
Holgate, World Allergy Organ J. 2014 Jul. 29; 7(1):17. doi: 10.1186/1939-4551-7-17. eCollection 2014. Review. PMID: 25097719
Incorvaia et al., Drug Des Devel Ther. 2014 Feb. 7; 8:197-207. doi: 10.2147/DDDT.S49409. eCollection 2014. PMID: 24532966
Inführ et al., Allergy. 2005 August; 60(8):977-85. Review. PMID: 15969677
Jawa et al., Clin Immunol. 2013 December; 149(3):534-55. doi: 10.1016/j.clim.2013.09.006. Epub 2013 Sep. 25. Review. PMID: 24263283
Kreutz M, et al. 2013; Blood: 121 (15): 2836-2844
Lauer et al., Gastroenterology, vol. 127, no. 3, pp. 924-936, 2004. PMID: 15362047
WO 2004/000217 A2
Lin et al., Mol Immunol. 2012 October; 52(3-4):190-9. doi: 10.1016/j.molimm.2012.05.015. Epub 2012 Jun. 15. PMID: 22706073
Liu et al., Nature 507, 519-522, 2014, doi: 10.1038/nature12978
Lobert V H, et al, EMBO Rep. 2013 January; 14(1): 10-11. doi: 10.1038/embor.2012.188
Lowe et al., Regul Toxicol Pharmacol. 2015 February; 71(1):68-77. doi: 10.1016/j.yrtph.2014.12.002. Epub 2014 Dec. 8. PMID: 25497995
Lundegaard et al., Nucleic Acids Res. 1; 36:W509-12. 2008), PREDEP (Protein Sci. 2000 September; 9(9):1838-46
McIntush E W, EMBO Rep. 2013 June; 14(6): 495-496. doi: 10.1038/embor.2013.68
Milgrom et al., N Engl J Med. 1999 Dec. 23; 341(26):1966-73. PMID: 10607813
Petermann et al., FEBS Lett. 2011 Dec. 1; 585(23):3747-57. doi: 10.1016/j.febslet.2011.03.064. Epub 2011 Apr. 6. Review. PMID: 21477588
Poggianella et al., J Immunol. 2006 Sep. 15; 177(6):3597-605. PMID: 16951319
Pride et al., Neurodegener Dis. 2008; 5(3-4):194-6. doi: 10.1159/000113700. Epub 2008 Mar. 6. Review. PMID: 18322388
Rammensee et al., Immunogenetics (1999) 50: 213-219)
Schueler-Furman et al., Protein Sci. 2000 September; 9(9):1838-46
Schwabe et al., Genetic Engineering & Biotechnology News. Vol. 28, No. 4, 2008.
Silva et al, Journal of Controlled Release 2013, 168(2):179-199.
Uchida et al J. Biol. Chem. 218; 3838-3844, 1973
Usatine et al., Am Fam Physician. 2010 Aug. 1; 82(3):249-255.
Weidle U H et al., Semin Oncol. 2014 October; 41(5):653-60. doi: 10.1053/j.seminoncol.2014.08.004.
Weidle U H et al., Cancer Genomics Proteomics. 2013 July-August; 10(4):155-68.
WO 1996/012740 A1
Wu et al., Curr Opin Immunol. 2014 December; 31:8-15. doi: 10.1016/j.coi.2014.08.001. Epub 2014 Aug. 25. PMID: 25156315

1: A vaccine, comprising at least one peptide bound to a pharmaceutically acceptable carrier, wherein said peptide is selected from the group consisting of:
(SEQ ID NO: 109)
QQQGLPRAAGG,
(SEQ ID NO: 110)
QQLGLPRAAGG,
(SEQ ID NO: 111)
QQQGLPRAAEG,
(SEQ ID NO: 112)
QQLGLPRAAEG,
(SEQ ID NO: 113)
QQQGLPRAAG,
(SEQ ID NO: 114)
QQLGLPRAAG,
(SEQ ID NO: 115)
QQQGLPRAAE,
(SEQ ID NO: 116)
QQLGLPRAAE,
(SEQ ID NO: 117)
HSGQQQGLPRAAGG,
(SEQ ID NO: 118)
HSGQQLGLPRAAGG,
(SEQ ID NO: 119)
HSGQQQGLPRAAEG,
(SEQ ID NO: 120)
HSGQQLGLPRAAEG,
(SEQ ID NO: 121)
QSQRAPDRVLCHSG,
(SEQ ID NO: 122)
GSAQSQRAPDRVL,
and
(SEQ ID NO: 125)
WPGPPELDV.
wherein the vaccine is suitable for use in the treatment of an Immunoglobulin E (IgE) related disease.

2: The vaccine according to claim 1, wherein the IgE-related disease is selected from the group consisting of:
an allergic disease,
an IgE related autoimmune disease,
an eosinophil-associated disease, and
a lymphoma.

3: The vaccine according to claim 1, wherein at least one cysteine residue is bound as a linker to the N- or C-terminus of the peptide.

4: The vaccine according to claim 1, wherein at least one cysteine residue is bound as a linker to the N-terminus of the peptide.

5: The vaccine according to claim 1, wherein the carrier is a protein carrier.

6: The vaccine according to claim 5, wherein the protein carrier is selected from the group consisting of keyhole limpet haemocyanin (KLH), Crm-197, tetanus toxoid (TT) and diphtheria toxin (DT).

7: The vaccine according to claim 1, wherein the vaccine is formulated with an adjuvant.

8: The vaccine according to claim 1, formulated for intravenous, subcutaneous, intradermal or intramuscular administration.

9: The vaccine according to claim 1, wherein the peptide is contained in the vaccine in an amount from 0.1 ng to 10 mg.

10: The vaccine according to claim 1, wherein the peptide is bound to the carrier by a linker.

11: The vaccine according to claim 10, wherein the linker is a peptide linker selected from the group consisting of:
Gly-Gly-Cys,
Gly-Gly,
Gly-Cys,
Cys-Gly, and
Cys-Gly-Gly.

12: The vaccine according to claim 1, comprising at least two peptides, wherein the vaccine comprises:
(a) one or more peptides according to claim 1 combined with one or more IgE peptides, or
(b) two or more peptides according to claim 1.

13: The vaccine according to claim 12, comprising:
(i) a peptide selected from the group consisting of:
(SEQ ID NO: 109)
QQQGLPRAAGG,
(SEQ ID NO: 110)
QQLGLPRAAGG,
(SEQ ID NO: 111)
QQQGLPRAAEG,
and
(SEQ ID NO: 112)
QQLGLPRAAEG,
and
(ii) a peptide selected from the group consisting of:
(SEQ ID NO: 121)
QSQRAPDRVLCHSG,
(SEQ ID NO: 122)
GSAQSQRAPDRVL,
(SEQ ID NO: 117)
HSGQQQGLPRAAGG,
and
(SEQ ID NO: 125)
WPGPPELDV

14: A peptide, optionally bound to a pharmaceutically acceptable carrier, wherein said peptide is selected from the group consisting of:
(SEQ ID NO: 109)
QQQGLPRAAGG,
(SEQ ID NO: 110)
QQLGLPRAAGG,
(SEQ ID NO: 111)
QQQGLPRAAEG,
(SEQ ID NO: 112)
QQLGLPRAAEG,
(SEQ ID NO: 113)
QQQGLPRAAG,
(SEQ ID NO: 114)
QQLGLPRAAG,
(SEQ ID NO: 115)
QQQGLPRAAE,
(SEQ ID NO: 116)
QQLGLPRAAE,
(SEQ ID NO: 117)
HSGQQQGLPRAAGG,
(SEQ ID NO: 118)
HSGQQLGLPRAAGG,
(SEQ ID NO: 119)
HSGQQQGLPRAAEG,
(SEQ ID NO: 120)
HSGQQLGLPRAAEG,
(SEQ ID NO: 121)
QSQRAPDRVLCHSG,
(SEQ ID NO: 122)
GSAQSQRAPDRVL,
and
(SEQ ID NO: 125)
WPGPPELDV.
